SEQUENCE LISTING (70 sequences)

SEQ ID NO: 1 - protein, 282 aa, synthetic construct
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 2 - protein, 278 aa, synthetic construct
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 3 - protein, 162 aa, synthetic construct
MGSPGMVLGLLVQIWDDPTQNRNRIASFPGFLFVLLGVGSMGVAAIVWGAWFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 4 - DNA, 849 bp, synthetic construct
  source 1..849: mol_type = other DNA
atggggtccccgggcatggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgtggggtgcctggttctggggccgccgcagctgccagcaaagggactcaggtaacagcccaggaaatgcattctacagcaacgtcctataccggccccggggggccccaaagaagagtgaggactgctctggagaggggaaggaccagaggggccagagcatttattcaacctccttcccgcaaccggccccccgccagccgcacctggcgtcaagaccctgccccagcccgagaccctgccccagccccaggcccggccaccccgtctctatggtcagggtctctcctagaccaagccccacccagcagccgaggccaaaagggttccccaaagtgggagaggagtga

SEQ ID NO: 5 - DNA, 837 bp, synthetic construct
  source 1..837: mol_type = other DNA
atggggtccccgggcatggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgtggggtgcctggttctggggccgccgcagctgccagcaaagggactcaggaaatgcattctacagcaacgtcctataccggccccggggggccccaaagaagagtgaggactgctctggagaggggaaggaccagaggggccagagcatttattcaacctccttcccgcaaccggccccccgccagccgcacctggcgtcaagaccctgccccagcccgagaccctgccccagccccaggcccggccaccccgtctctatggtcagggtctctcctagaccaagccccacccagcagccgaggccaaaagggttccccaaagtgggagaggagtga

SEQ ID NO: 6 - DNA, 489 bp, synthetic construct
  source 1..489: mol_type = other DNA
atggggtccccgggcatggtgctgggcctcctggtgcagatctgggatgaccccacacagaacagaaaccggatcgcaagcttcccaggattcctcttcgtgctgctgggggtgggaagcatgggtgtggctgcgatcgtgtggggtgcctggttctggggccgccgcagctgccagcaaagggactcaggtaacagcccaggaaatgcattctacagcaacgtcctataccggccccggggggccccaaagaagagtgaggactgctctggagaggggaaggaccagaggggccagagcatttattcaacctccttcccgcaaccggccccccgccagccgcacctggcgtcaagaccctgccccagcccgagaccctgccccagccccaggcccggccaccccgtctctatggtcagggtctctcctagaccaagccccacccagcagccgaggccaaaagggttccccaaagtgggagaggagtga

SEQ ID NO: 7 - DNA, 354 bp, synthetic construct
  source 1..354: mol_type = other DNA
gaggttcagctgcagcagtctggacctgagctggtgaagcctgggactttagtgaatatatcctgcaaggcttctggttacaccttcacaagctacgatataaactgggtgaagcagaggcctggacagggacttgagtggattggatggatttatcctggagatggtagtactaactacaatgagaaattcaagggcaaggccacactgactgcagacaaatcctccagcacagcctacatgcagctcagcagcctgacttctgagaactctgcagtctatttctgtgcaagaagagggctacggtactactttgactactggggccaaggcaccactctcacagtctcctca

SEQ ID NO: 8 - DNA, 30 bp, synthetic construct
  source 1..30: mol_type = other DNA
ggttacaccttcacaagctacgatataaac

SEQ ID NO: 9 - DNA, 51 bp, synthetic construct
  source 1..51: mol_type = other DNA
tggatttatcctggagatggtagtactaactacaatgagaaattcaagggc

SEQ ID NO: 10 - DNA, 33 bp, synthetic construct
  source 1..33: mol_type = other DNA
gcaagaagagggctacggtactactttgactac

SEQ ID NO: 11 - DNA, 75 bp, synthetic construct
  source 1..75: mol_type = other DNA
gaggttcagctgcagcagtctggacctgagctggtgaagcctgggactttagtgaatatatcctgcaaggcttct

SEQ ID NO: 12 - DNA, 42 bp, synthetic construct
  source 1..42: mol_type = other DNA
tgggtgaagcagaggcctggacagggacttgagtggattgga

SEQ ID NO: 13 - DNA, 90 bp, synthetic construct
  source 1..90: mol_type = other DNA
aaggccacactgactgcagacaaatcctccagcacagcctacatgcagctcagcagcctgacttctgagaactctgcagtctatttctgt

SEQ ID NO: 14 - DNA, 33 bp, synthetic construct
  source 1..33: mol_type = other DNA
tggggccaaggcaccactctcacagtctcctca

SEQ ID NO: 15 - DNA, 321 bp, synthetic construct
  source 1..321: mol_type = other DNA
gatatccagatgacacagactacatcctccctgtctgcctctctgggagacagagtcaccattagttgcagggcaagtcaggacattaggaattatttaaactggtatcagcagaaaccagatggcactgttaaactcctgatctactacacatcaagattacattcaggagtcccatcaaggttcagtggcagtgggtctggaacagattattctctcaccattagcaacctggagcaagaagatattgccacttacttttgccaacaggttaatacgcttccgtggacgttcggtggaggcaccaagctggaaatcaaa

SEQ ID NO: 16 - DNA, 33 bp, synthetic construct
  source 1..33: mol_type = other DNA
agggcaagtcaggacattaggaattatttaaac

SEQ ID NO: 17 - DNA, 21 bp, synthetic construct
  source 1..21: mol_type = other DNA
tacacatcaagattacattca

SEQ ID NO: 18 - DNA, 27 bp, synthetic construct
  source 1..27: mol_type = other DNA
caacaggttaatacgcttccgtggacg

SEQ ID NO: 19 - DNA, 69 bp, synthetic construct
  source 1..69: mol_type = other DNA
gatatccagatgacacagactacatcctccctgtctgcctctctgggagacagagtcaccattagttgc

SEQ ID NO: 20 - DNA, 45 bp, synthetic construct
  source 1..45: mol_type = other DNA
tggtatcagcagaaaccagatggcactgttaaactcctgatctac

SEQ ID NO: 21 - DNA, 96 bp, synthetic construct
  source 1..96: mol_type = other DNA
ggagtcccatcaaggttcagtggcagtgggtctggaacagattattctctcaccattagcaacctggagcaagaagatattgccacttacttttgc

SEQ ID NO: 22 - DNA, 30 bp, synthetic construct
  source 1..30: mol_type = other DNA
ttcggtggaggcaccaagctggaaatcaaa

SEQ ID NO: 23 - protein, 118 aa, synthetic construct
EVQLQQSGPELVKPGTLVNISCKASGYTFTSYDINWVKQRPGQGLEWIGWIYPGDGSTNYNEKFKGKATLTADKSSSTAYMQLSSLTSENSAVYFCARRGLRYYFDYWGQGTTLTVSS

SEQ ID NO: 24 - protein, 10 aa, synthetic construct
GYTFTSYDIN

SEQ ID NO: 25 - protein, 17 aa, synthetic construct
WIYPGDGSTNYNEKFKG

SEQ ID NO: 26 - protein, 11 aa, synthetic construct
ARRGLRYYFDY

SEQ ID NO: 27 - protein, 25 aa, synthetic construct
EVQLQQSGPELVKPGTLVNISCKAS

SEQ ID NO: 28 - protein, 14 aa, synthetic construct
WVKQRPGQGLEWIG

SEQ ID NO: 29 - protein, 30 aa, synthetic construct
KATLTADKSSSTAYMQLSSLTSENSAVYFC

SEQ ID NO: 30 - protein, 11 aa, synthetic construct
WGQGTTLTVSS

SEQ ID NO: 31 - protein, 107 aa, synthetic construct
DIQMTQTTSSLSASLGDRVTISCRASQDIRNYLNWYQQKPDGTVKLLIYYTSRLHSGVPSRFSGSGSGTDYSLTISNLEQEDIATYFCQQVNTLPWTFGGGTKLEIK

SEQ ID NO: 32 - protein, 11 aa, synthetic construct
RASQDIRNYLN

SEQ ID NO: 33 - protein, 7 aa, synthetic construct
YTSRLHS

SEQ ID NO: 34 - protein, 9 aa, synthetic construct
QQVNTLPWT

SEQ ID NO: 35 - protein, 23 aa, synthetic construct
DIQMTQTTSSLSASLGDRVTISC

SEQ ID NO: 36 - protein, 15 aa, synthetic construct
WYQQKPDGTVKLLIY

SEQ ID NO: 37 - protein, 32 aa, synthetic construct
GVPSRFSGSGSGTDYSLTISNLEQEDIATYFC

SEQ ID NO: 38 - protein, 10 aa, synthetic construct
FGGGTKLEIK

SEQ ID NO: 39 - DNA, 354 bp, synthetic construct
  source 1..354: mol_type = other DNA
cagctgaagcagtcaggacctggcctggtgaaaccttctcagtcactgtccctcacctgcactgtcactggctactcaatcaccagtgattatgcctggaactggatccggcagtttccaggaaacaaactggagttgatgggctacataacctacagtggtagcactagctacaacccatctctcaaaagtcgattctctatcactcgagacacatccaagaaccagttcttcctgcagttgaattctgtgactactgaggacacagccacatattactgtgcaagatcggggtataggtacgacgatgctatggactactggggtcaaggaacctcagtcaccgtctcctca

SEQ ID NO: 40 - DNA, 33 bp, synthetic construct
  source 1..33: mol_type = other DNA
ggctactcaatcaccagtgattatgcctggaac

SEQ ID NO: 41 - DNA, 48 bp, synthetic construct
  source 1..48: mol_type = other DNA
tacataacctacagtggtagcactagctacaacccatctctcaaaagt

SEQ ID NO: 42 - DNA, 39 bp, synthetic construct
  source 1..39: mol_type = other DNA
gcaagatcggggtataggtacgacgatgctatggactac

SEQ ID NO: 43 - DNA, 69 bp, synthetic construct
  source 1..69: mol_type = other DNA
cagctgaagcagtcaggacctggcctggtgaaaccttctcagtcactgtccctcacctgcactgtcact

SEQ ID NO: 44 - DNA, 42 bp, synthetic construct
  source 1..42: mol_type = other DNA
tggatccggcagtttccaggaaacaaactggagttgatgggc

SEQ ID NO: 45 - DNA, 90 bp, synthetic construct
  source 1..90: mol_type = other DNA
cgattctctatcactcgagacacatccaagaaccagttcttcctgcagttgaattctgtgactactgaggacacagccacatattactgt

SEQ ID NO: 46 - DNA, 33 bp, synthetic construct
  source 1..33: mol_type = other DNA
tggggtcaaggaacctcagtcaccgtctcctca

SEQ ID NO: 47 - DNA, 339 bp, synthetic construct
  source 1..339: mol_type = other DNA
gacattgtgatgacacagtctccatcctccctggctatgtcagtcggacagaaggtcactatgagctgcaagtccagtcagagccttttaagtagtaataatcaaaagaactatttggcctggtaccagctgaaaccaggacagtctcctaaacttctggtatattttgcatccactagggaatctggggtccctgatcgcttcataggcagtggatctgggacagatttcactcttactatcagcagtgtgcaggctgaagacctggcagattacttctgtcagcaacattatcgcactccgctcacgttcggtgctgggaccaagctggagctgaaa

SEQ ID NO: 48 - DNA, 51 bp, synthetic construct
  source 1..51: mol_type = other DNA
aagtccagtcagagccttttaagtagtaataatcaaaagaactatttggcc

SEQ ID NO: 49 - DNA, 21 bp, synthetic construct
  source 1..21: mol_type = other DNA
tttgcatccactagggaatct

SEQ ID NO: 50 - DNA, 27 bp, synthetic construct
  source 1..27: mol_type = other DNA
cagcaacattatcgcactccgctcacg

SEQ ID NO: 51 - DNA, 69 bp, synthetic construct
  source 1..69: mol_type = other DNA
gacattgtgatgacacagtctccatcctccctggctatgtcagtcggacagaaggtcactatgagctgc

SEQ ID NO: 52 - DNA, 45 bp, synthetic construct
  source 1..45: mol_type = other DNA
tggtaccagctgaaaccaggacagtctcctaaacttctggtatat

SEQ ID NO: 53 - DNA, 96 bp, synthetic construct
  source 1..96: mol_type = other DNA
ggggtccctgatcgcttcataggcagtggatctgggacagatttcactcttactatcagcagtgtgcaggctgaagacctggcagattacttctgt

SEQ ID NO: 54 - DNA, 30 bp, synthetic construct
  source 1..30: mol_type = other DNA
ttcggtgctgggaccaagctggagctgaaa

SEQ ID NO: 55 - protein, 118 aa, synthetic construct
QLKQSGPGLVKPSQSLSLTCTVTGYSITSDYAWNWIRQFPGNKLELMGYITYSGSTSYNPSLKSRFSITRDTSKNQFFLQLNSVTTEDTATYYCARSGYRYDDAMDYWGQGTSVTVSS

SEQ ID NO: 56 - protein, 11 aa, synthetic construct
GYSITSDYAWN

SEQ ID NO: 57 - protein, 16 aa, synthetic construct
YITYSGSTSYNPSLKS

SEQ ID NO: 58 - protein, 13 aa, synthetic construct
ARSGYRYDDAMDY

SEQ ID NO: 59 - protein, 23 aa, synthetic construct
QLKQSGPGLVKPSQSLSLTCTVT

SEQ ID NO: 60 - protein, 14 aa, synthetic construct
WIRQFPGNKLELMG

SEQ ID NO: 61 - protein, 30 aa, synthetic construct
RFSITRDTSKNQFFLQLNSVTTEDTATYYC

SEQ ID NO: 62 - protein, 11 aa, synthetic construct
WGQGTSVTVSS

SEQ ID NO: 63 - protein, 113 aa, synthetic construct
DIVMTQSPSSLAMSVGQKVTMSCKSSQSLLSSNNQKNYLAWYQLKPGQSPKLLVYFASTRESGVPDRFIGSGSGTDFTLTISSVQAEDLADYFCQQHYRTPLTFGAGTKLELK

SEQ ID NO: 64 - protein, 17 aa, synthetic construct
KSSQSLLSSNNQKNYLA

SEQ ID NO: 65 - protein, 7 aa, synthetic construct
FASTRES

SEQ ID NO: 66 - protein, 9 aa, synthetic construct
QQHYRTPLT

SEQ ID NO: 67 - protein, 23 aa, synthetic construct
DIVMTQSPSSLAMSVGQKVTMSC

SEQ ID NO: 68 - protein, 15 aa, synthetic construct
WYQLKPGQSPKLLVY

SEQ ID NO: 69 - protein, 32 aa, synthetic construct
GVPDRFIGSGSGTDFTLTISSVQAEDLADYFC

SEQ ID NO: 70 - protein, 10 aa, synthetic construct
FGAGTKLELK

(19) United States
(12) Patent Application Publication
(10) Pub. No.: US 2024/0336917 A1
(43) Pub. Date: Oct. 10, 2024

(54) COMPOSITIONS AND METHODS FOR INHIBITING THE EXPRESSION OF TMIGD2

(71) Applicant: ALBERT EINSTEIN COLLEGE OF MEDICINE, Bronx, NY (US)

(72) Inventors: Xingxing ZANG, Bronx, NY (US); Hao WANG, Bronx, NY (US); Roberto Alejandro SICA, Bronx, NY (US)

(21) Appl. No.: 18/573,156

(22) PCT Filed: Jul. 1, 2022
(86) PCT No.: PCT/US2022/073387

§ 371 Date: Dec. 21, 2023

Related U.S. Application Data
(60) Provisional application No. 63/217,630, filed on Jul. 1, 2021.

(51) Int. Cl. C12N 15/113; A61K 39/00; A61P 35/02; C07K 16/28; C12N 9/22; C12N 15/11

(52) U.S. Cl. CPC C12N 15/113; A61P 35/02; C07K 16/2818; C12N 9/22; C12N 15/111; A61K 2039/505; C07K 2317/56; C07K 2317/76; C12N 2310/11; C12N 2310/14; C12N 2310/20

(57) ABSTRACT

Methods of treating cancer by inhibiting TMIGD2 expression and/or activity are provided herein. In some embodiments, the methods comprise administering one or more of: (a) a TMIGD2 mRNA targeting agent, (b) a gene-based therapeutic agent, (c) a small molecule TMIGD2 inhibitory molecule, or (d) a TMIGD2 antibody or antigen-binding fragment thereof to inhibit TMIGD2 expression and/or activity in a subject in need thereof.

### PRIORITY CLAIM

This application claims priority to U.S. Provisional Patent Application No. 63/217,630, filed Jul. 1, 2021, the content of which is hereby incorporated by reference in its entirety.

### STATEMENT REGARDING FEDERAL FUNDING

This invention was made with government support under R01CA175495 awarded by the National Institutes of Health. The government has certain rights in this invention.

### SEQUENCE LISTING

This application contains an ST.26 compliant Sequence Listing, which was submitted in xml format via EFS-Web and is hereby incorporated by reference in its entirety. The .xml copy, created on Jul. 1, 2022 is named SequenceListing.xml and is 66.1 KB in size.

### BACKGROUND

Cancer is a serious public health problem in the U.S. and other countries. More than 90% of cancer patient deaths result from cancer metastasis rather than from a primary cancer. There are about 924,310 new cancer cases and 339, 150 cancer deaths in the U.S. alone. According to Cancer Statistics 2010, in 2010 alone in the U.S. there were an estimated 222,520 new cases and 157,300 deaths for lung cancer, 217,730 new cases and 32,050 deaths for prostate cancer, 207,090 new cases and 39,840 deaths for breast cancer, 145,500 new cases and 51,370 deaths for gut cancer, 58,240 new cases and 8,210 deaths for kidney cancer, and 51,350 new cases and 36,800 deaths for pancreatic cancer. While traditional therapies such as surgery, chemotherapy, and radiation can often control primary cancer growth, successful control of cancer remains rare.

As such, there is a serious and long-felt need for the development of cancer treatments with increased efficacy.

### SUMMARY

Aspects of the present disclosure provide methods of treating or preventing cancer in a subject in need thereof, the methods comprising administering to the subject an effective amount of an agent that inhibits TMIGD2 expression, activity, or both.

In some embodiments, the agent is selected from the group consisting of an antibody agent, a mRNA targeting agent, a small molecule agent, and a gene editing agent.

In some embodiments, the mRNA targeting agent is an antisense agent or an RNAi agent. In some embodiments, the antisense agent comprises or consists of a nucleic acid sequence complementary to an mRNA encoded by SEQ ID NO:4, SEQ ID NO:5, or SEQ ID NO:6, or the antisense agent comprises or consists of a nucleic acid sequence with about 80%, 90%, 95%, 96%, 97%, 98%, 99%, 99.5%, or higher identity to an mRNA encoded by SEQ ID NO:4, SEQ ID NO:5, or SEQ ID NO:6.

In some embodiments, the RNAi agent is selected from the group consisting of a short interfering RNA (siRNA), a double-stranded RNA (dsRNA), a micro-RNA (miRNA), a piwi-RNA (piRNA), a small nucleolar RNA (snoRNA), a tRNA-derived small RNAs (tsRNAs), a small regulatory RNA (srRNA), and a short hairpin RNA (shRNA) molecule. In some embodiments, the RNAi agent comprises or consists of a nucleic acid sequence complementary to an mRNA encoded by SEQ ID NO:4, SEQ ID NO:5, or SEQ ID NO:6, or the RNAi agent comprises or consists of a nucleic acid sequence with about 80%, 90%, 95%, 96%, 97%, 98%, 99%, 99.5% or higher identity to an mRNA encoded by SEQ ID NO:4, SEQ ID NO:5, or SEQ ID NO:6.

In some embodiments, the gene editing agent is selected from the group consisting of a TALEN-based agent, ZFN-based agent, and a CRISPR-based agent. In some embodiments, the gene editing agent knocks out or knocks down expression of TMIGD2.

In some embodiments, the antibody agent is an antibody or antigen-binding fragment thereof that specifically binds an epitope in the extracellular domain of TMIGD2. In some embodiments, the extracellular domain of TMIGD2 comprises residues 1-150 of SEQ ID NO: 1 or SEQ ID NO:2, or residues 1-30 of SEQ ID NO:3.

In some embodiments, an antibody or antigen-binding fragment thereof comprises: (a) a heavy chain variable region comprising: GYTFTSYDIN (SEQ ID NO: 24), WIYPGDGSTNYNEKFKG (SEQ ID NO: 25), and/or ARRGLRYYFDY (SEQ ID NO: 26); and (b) a light chain variable region comprising: RASQDIRNYLN (SEQ ID NO: 32), YTSRLHS (SEQ ID NO: 33), and QQVNTLPWT (SEQ ID NO: 34). In some embodiments, an antibody or antigen-binding fragment thereof comprises: (a) a heavy chain variable region comprising: GYSITSDYAWN (SEQ ID NO: 56). YITYSGSTSYNPSLKS (SEQ ID NO: 57), and/or ARSGYRYDDAMDY (SEQ ID NO: 58); and (b) a light chain variable region comprising: KSSQSLLSSNNQKNYLA (SEQ ID NO: 64), FASTRES (SEQ ID NO: 65), and QQHYRTPLT (SEQ ID NO: 66).

In some embodiments, an antibody or antigen-binding fragment thereof comprises: (a) a heavy chain variable region comprising SEQ ID NO: 23, or an amino acid sequence with at least 85%, 90%, 95%, 99%, or higher identity to SEQ ID NO: 23; and/or (b) a light chain variable region comprising SEQ ID NO: 31, or an amino acid sequence with at least 85%, 90%, 95%, 99%, or higher identity to SEQ ID NO: 31. In some embodiments, an antibody or antigen-binding fragment thereof comprises: (a) a heavy chain variable region comprising SEQ ID NO: 55, or an amino acid sequence with at least 85%, 90%, 95%, 99%, or higher identity to SEQ ID NO: 25; and/or (b) a light chain variable region comprising SEQ ID NO: 63, or an amino acid sequence with at least 85%, 90%, 95%, 99%, or higher identity to SEQ ID NO: 63.

In some embodiments, the cancer is a human hematologic malignancy. In some embodiments, the human hematologic malignancy is selected from myeloid neoplasm, acute myeloid leukemia (AML), AML with recurrent genetic abnormalities, AML with myelodysplasia-related changes, therapy-related AML, acute leukemias of ambiguous lineage, myeloproliferative neoplasm, essential thrombocythemia, polycythemia vera, myelofibrosis (MF), primary myelofibrosis, systemic mastocytosis, myelodysplastic syndromes (MDS), myeloproliferative/myelodysplastic syndromes, chronic myeloid leukemia, chronic neutrophilic leukemia, chronic eosinophilic leukemia, myelodysplastic syndromes (MDS), refractory anemia with ringed sideroblasts, refractory cytopenia with multilineage dysplasia, refractory anemia with excess blasts (type 1), refractory anemia with excess blasts (type 2), MDS with isolated del (5q), unclassifiable MDS, myeloproliferative/myelodysplastic syndromes, chronic myelomonocytic leukemia, atypical chronic myeloid leukemia, juvenile myelomonocytic leukemia, unclassifiable myeloproliferative/myelodysplatic syndromes, lymphoid neoplasms, precursor lymphoid neoplasms, B lymphoblastic leukemia, B lymphoblastic lymphoma, T lymphoblastic leukemia, T lymphoblastic lymphoma, mature B-cell neoplasms, diffuse large B-cell lymphoma, primary central nervous system lymphoma, primary mediastinal B-cell lymphoma, Burkitt lymphoma/leukemia, follicular lymphoma, chronic lymphocytic leukemia, small lymphocytic lymphoma, B-cell prolymphocytic leukemia, lymphoplasmacytic lymphoma, Waldenström macroglobulinemia, mantle cell lymphoma, marginal zone lymphomas, post-transplant lymphoproliferative disorders, HIV-associated lymphomas, primary effusion lymphoma, intravascular large B-cell lymphoma, primary cutaneous B-cell lymphoma, hairy cell leukemia, multiple myeloma, monoclonal gammopathy of unknown significance (MGUS), smoldering multiple myeloma, or solitary plasmacytomas (solitary bone and extramedullary).

In another aspect, the disclosure provides anti-TMIGD2 antibodies or antigen-binding fragments thereof. In some embodiments, an antibody or antigen-binding fragment thereof described herein comprises: a) a heavy chain variable region comprising: GYTFTSYDIN (SEQ ID NO: 24), WIYPGDGSTNYNEKFKG (SEQ ID NO: 25), and ARRGLRYYFDY (SEQ ID NO: 26); and a light chain variable region comprising: RASQDIRNYLN (SEQ ID NO: 32), YTSRLHS (SEQ ID NO: 33), and QQVNTLPWT (SEQ ID NO: 34); or (b) a heavy chain variable region comprising: GYSITSDYAWN (SEQ ID NO: 56). YITYSGSTSYNPSLKS (SEQ ID NO: 57), and ARSGYRYDDAMDY (SEQ ID NO: 58); and a light chain variable region comprising: KSSQSLLSSNNQKNYLA (SEQ ID NO: 64), FASTRES (SEQ ID NO: 65), and QQHYRTPLT (SEQ ID NO: 66). In some embodiments, an antibody or antigen-binding fragment thereof described herein comprises: (a) a heavy chain variable region comprising SEQ ID NO: 23, or an amino acid sequence with at least 85%, 90%, 95%, 99%, or higher identity to SEQ ID NO: 23; and (b) a light chain variable region comprising SEQ ID NO: 31, or an amino acid sequence with at least 85%, 90%, 95%, 99%, or higher identity to SEQ ID NO: 31. In some embodiments, an antibody or antigen-binding fragment thereof described herein comprises: (a) a heavy chain variable region comprising SEQ ID NO: 55, or an amino acid sequence with at least 85%, 90%, 95%, 99%, or higher identity to SEQ ID NO: 25; and (b) a light chain variable region comprising SEQ ID NO: 63, or an amino acid sequence with at least 85%, 90%, 95%, 99%, or higher identity to SEQ ID NO: 63.

### BRIEF DESCRIPTION OF THE DRAWINGS

FIGS. 1A-1B is a representative plot showing that TMIGD2 is highly expressed on various human hematologic malignancies including human erythroleukemia (HEL), chronic myelogenous leukemia (CML), and acute myelogenous leukemia (Kg1a) analyzed by flow cytometry (FIG. 1A). Anti-TMIGD2 mAb (open histograms) and isotype control (shaded histograms) are shown. Using the Cancer Cell Line Encyclopedia (CCLE) and Genevestigator, TMIGD2 mRNA was highly expressed in cell lines of human leukemia, lymphoma, multiple myeloma, etc. (FIG. 1B).

FIGS. 2A-2C are representative plots shows that TMIGD2 mRNA, but not PD-L1/PD-1, is highly expressed in human AML and is associated with worse overall survival of patients (FIGS. 2A-2B). The mRNA levels of the newly identified HHLA2/TMIGD2/KIR3DL3 pathways and the long-standing PD-L1/PD-1 pathway in TCGA and GTEx datasets of human AML were analyzed using Gene Expression Profiling Interactive Analysis. Tumor=173, Normal=70; * P<0.05. (FIG. 2C). The TMIGD2 high group (top 25%) indicated showed a significant worse overall survival than the TMIGD2 low group (the rest 75%) in AML patients. p=0.011 (FIG. 2C).

FIGS. 3A-3B are representative plots comparing the expression levels of TMIGD2 on AML stem/progenitor cells to AML differentiated blasts (FIG. 3A) or CD34+ normal stem/progenitor cells in cord blood/adult bone marrow mononuclear cells from healthy donors (FIG. 3B). N=40 AML patients, 10 healthy donors. ** P<0.01, **** P<0.0001, determined by a two-sided paired (FIG. 3A) or unpaired (FIG. 3B) Student's t test. Mean values are shown unless otherwise specified, and error bars represent±SEM.

FIGS. 4A-4D are representative plots showing that TMIGD2 is enriched in functional leukemia-initiating cells. Schematic for flow cytometry sorting TMIGD2+ and TMIGD2-AML stem cells and subsequent colony-forming unit (CFU) assay and in vivo limiting dilution xenotransplantation assay (FIG. 4A). Results of first round and second round CFU assays using TMIGD2+ or TMIGD2-primary AML stem cells from patient #31 and #27 (FIG. 4B). Leukemic engraftment in irradiated NSG mice transplanted with TMIGD2+ and TMIGD2-AML stem cells from patient #31 (FIG. 4C). Transcriptome-wide RNA-sequencing (RNA-seq) was conducted on flow-sorted CD34+TMIGD2+ and CD34+TMIGD2-fractions of six primary AML specimens. Gene set enrichment analysis (GSEA) showed that the top seven pathways enriched in CD34+TMIGD2+ fraction consisted of E2F targets, MYC targets, and G2M checkpoints, which was consistent with the findings that CD34+TMIGD2+ cells generated more colonies and induced leukemia much more efficiently than TMIGD2-counterparts (FIG. 4D). Moreover, CD34+TMIGD2+ subpopulation was associated with the established leukemic stem cells (LSC) and 17-gene stemness signatures, while corresponding TMIGD2-fraction was correlated with myeloid cell development, hematopoiesis maturation, and downregulation of HOXA9 and MEIS1 targets (FIG. 4D).

FIGS. 5A-5D are representative plots showing that the loss or blocking of TMIGD2 impairs AML stem cells maintenance. In particular, the plots include a schematic for flow cytometry sorting strategy for TMIGD2+AML stem cells and lentivirus transduction (FIG. 5A), FACS examination of TMIGD2 expression on AML stem cells transduced with lentivirus expressing Scramble control shRNA (shCtrl) or TMIGD2 specific shRNA (shTMIGD2) (FIG. 5B), and quantification of colony-forming unit results from three AML patients (FIG. 5C), N=3 independent experiments with shCtrl, shTMIGD2 #2, and shTMIGD2 #3. The therapeutic efficacy of anti-TMIGD2 mAbs 17C7 and 20F2 was assessed in vivo using clinically relevant AML patient-derived-xenograft (PDX) of various AML subtypes. The anti-leukemic effect of 17C7 and 20F2 anti-TMIGD2 mAbs was confirmed by the reduction of human CD45+ cells (AML cells) in peripheral blood and bone marrow following treatment (FIG. 5D). * P<0.05, ** P<0.01, *** P<0.001, **** P<0.0001, determined by a two-sided unpaired Student's t test. Mean values are shown unless otherwise specified, and error bars represent±SEM.

FIGS. 6A-6B are representative plots showing that knock-down of TMIGD2 increase cell death of human hematologic malignancies. In particular, the plots include apoptotic analysis of shCtrl and shTMIGD2 #3 HEL cells. Early apoptosis, Annexin V+DAPI-. Late apoptosis/necrosis, Annexin V+DAPI+ (FIG. 6B) and a representative heatmap showing genes enriched in apoptosis and cell cycle arrest (FIG. 6C). * p<0.05, ** p<0.01, *** p<0.001, determined by a two-sided unpaired Student's t test. Mean values are shown unless otherwise specified, and error bars represent±SEM.

### DETAILED DESCRIPTION

The B7 ligand family binds to the CD28 receptor family on T cells and other immune cells, which critically regulate functions of immune cells^{1,2}. The B7/CD28 pathways are attractive therapeutic targets and the FDA has approved several drugs developed from the B7/CD28 families.³⁻⁶ HERV-H LTR-associating protein 2 (HHLA2) was discovered as a new functional member of the B7 family in 2013⁷, which subsequently led to the discovery of immunoglobulin domain-containing protein 2 (TMIGD2) as a new member of the CD28 family and a receptor for HHLA2^{8,9}. TMIGD2 is expressed on T cells and NK cells and shows co-stimulatory function for T cells and NK cells^{1,10,11}. There are at least three isoforms of TMIGD2: isoform 1 (SEQ ID NO:1, NCBI NP_653216.2), isoform 2 (SEQ ID NO:2, NCBI NP_001162597.1), and isoform 3 (SEQ ID NO:3, NCBI NP_001295161.1). Exemplary DNA sequences encoding isoforms 1-3 are set forth in SEQ ID NO:4 (NCBI NM_144615), SEQ ID NO: 5 (NCBI NM_001169126.1), and SEQ ID NO:6 (NCBI NM_001308232), respectively.

TABLE 1
TMIGD2 Amino Acid Sequences

| SEQ | | |
|---|---|---|
| ID NO. | NAME | SEQUENCE |
| 1 | TMIGD2- | MGSPGMVLGLLVQIWALQEASSLSVQQGPNLLQVRQGSQ |
| | Isoform 1* | ATLVCQVDQATAWERLRVKWTKDGAILCQPYITNGSLSLG |
| | | VCGPQGRLSWQAPSHLTLQLDPVSLNHSGAYVCWAAVEI |
| | | PELEEAEGNITRLFVDPDDPTQNRNRIASFPGFLFVLLGVG |
| | | SMGVAAIVWGAWFWGRRSCQQRDSGNSPGNAFYSNVL |
| | | YRPRGAPKKSEDCSGEGKDQRGQSIYSTSFPQPAPRQPH |
| | | LASRPCPSPRPCPSPRPGHPVSMVRVSPRPSPTQQPRPK |
| | | GFPKVGEE |
| 2 | TMIGD2- | MGSPGMVLGLLVQIWALQEASSLSVQQGPNLLQVRQGSQ |
| | Isoform 2* | ATLVCQVDQATAWERLRVKWTKDGAILCQPYITNGSLSLG |
| | | VCGPQGRLSWQAPSHLTLQLDPVSLNHSGAYVCWAAVEI |
| | | PELEEAEGNITRLFVDPDDPTQNRNRIASFPGFLFVLLGVG |
| | | SMGVAAIVWGAWFWGRRSCQQRDSGNAFYSNVLYRPR |
| | | GAPKKSEDCSGEGKDQRGQSIYSTSFPQPAPRQPHLASR |
| | | PCPSPRPCPSPRPGHPVSMVRVSPRPSPTQQPRPKGFP |
| | | KVGEE |
| 3 | TMIGD2- | MGSPGMVLGLLVQIWDDPTQNRNRIASFPGFLFVLLGVG |
| | Isoform 3* | SMGVAAIVWGAWFWGRRSCQQRDSGNSPGNAFYSNVL |
| | | YRPRGAPKKSEDCSGEGKDQRGQSIYSTSFPQPAPRQPH |
| | | LASRPCPSPRPCPSPRPGHPVSMVRVSPRPSPTQQPRPK |
| | | GFPKVGEE |

*Amino acids corresponding to the intracellular regions are underlined, transmembrane regions are bolded, and extracellular regions are italicized of TMIDG2 isoforms 1, 2, and 3.

TABLE 2
TMIGD2 Nucleic Acid Sequences

| SEQ | | |
|---|---|---|
| ID NO. | NAME | SEQUENCE |
| 4 | TMIGD2- | atggggtccccgggcatggtgctgggcctcctggtgcagatctgggccctgcaag |
| | Isoform 1* | aagcctcaagcctgagcgtgcagcaggggcccaacttgctgcaggtgaggcag |
| | | ggcagtcaggcgaccctggtctgccaggtggaccaggccacagcctgggaacg |
| | | gctccgtgttaagtggacaaaggatggggccatcctgtgtcaaccgtacatcacc |
| | | aacggcagcctcagcctgggggtctgcgggccccagggacggctctcctggca |
| | | ggcacccagccatctcaccctgcagctggaccctgtgagcctcaaccacagcgg |
| | | ggcgtacgtgtgctgggcggccgtagagattcctgagttggaggaggctgaggg |
| | | caacataacaaggctctttgtggacccagatgaccccacacagaacagaaacc |
| | | ggatcgcaagcttcccaggattcctcttcgtgctgctgggggtgggaagcatg |
| | | ggtgtggctgcgatcgtgtggggtgcctggttctggggccgccgcagctgcc |
| | | agcaaagggactcaggtaacagcccaggaaatgcattctacagcaacgtcctat |
| | | accggccccggggggccccaaagaagagtgaggactgctctggagagggga |
| | | aggaccagaggggccagagcatttattcaacctccttcccgcaaccggcccccc |
| | | gccagccgcacctggcgtcaagaccctgccccagcccgagaccctgccccagc |
| | | cccaggcccggccaccccgtctctatggtcagggtctctcctagaccaagcccca |
| | | cccagcagccgaggccaaaagggttccccaaagtgggagaggagtga |
| 5 | TMIGD2- | atggggtccccgggcatggtgctgggcctcctggtgcagatctgggccctgcaag |
| | Isoform 2* | aagcctcaagcctgagcgtgcagcaggggcccaacttgctgcaggtgaggcag |
| | | ggcagtcaggcgaccctggtctgccaggtggaccaggccacagcctgggaacg |
| | | gctccgtgttaagtggacaaaggatggggccatcctgtgtcaaccgtacatcacc |
| | | aacggcagcctcagcctgggggtctgcgggccccagggacggctctcctggca |
| | | ggcacccagccatctcaccctgcagctggaccctgtgagcctcaaccacagcgg |
| | | ggcgtacgtgtgctgggcggccgtagagattcctgagttggaggaggctgaggg |
| | | caacataacaaggctctttgtggacccagatgaccccacacagaacagaaacc |
| | | ggatcgcaagcttcccaggattcctcttcgtgctgctgggggtgggaagcatg |
| | | ggtgtggctgcgatcgtgtggggtgcctggttctggggccgccgcagctgcc |
| | | agcaaagggactcaggaaatgcattctacagcaacgtcctataccggccccggg |
| | | gggccccaaagaagagtgaggactgctctggagaggggaaggaccagaggg |
| | | gccagagcatttattcaacctccttcccgcaaccggccccccgccagccgcacct |
| | | ggcgtcaagaccctgccccagcccgagaccctgccccagccccaggcccggc |
| | | caccccgtctctatggtcagggtctctcctagaccaagccccacccagcagccga |
| | | ggccaaaagggttccccaaagtgggagaggagtga |
| 6 | TMIGD2- | atggggtccccgggcatggtgctgggcctcctggtgcagatctgggatgacccca |
| | Isoform 3* | cacagaacagaaaccggatcgcaagcttcccaggattcctcttcgtgctgctg |
| | | ggggtgggaagcatgggtgtggctgcgatcgtgtggggtgcctggttctgg |
| | | ggccgccgcagctgccagcaaagggactcaggtaacagcccaggaaatgcatt |
| | | ctacagcaacgtcctataccggccccggggggccccaaagaagagtgaggact |
| | | gctctggagaggggaaggaccagaggggccagagcatttattcaacctccttcc |
| | | cgcaaccggccccccgccagccgcacctggcgtcaagaccctgccccagccc |
| | | gagaccctgccccagccccaggcccggccaccccgtctctatggtcagggtctct |
| | | cctagaccaagccccacccagcagccgaggccaaaagggttccccaaagtgg |
| | | gagaggagtga |

*Nucleic acids encoding the intracellular regions are underlined, transmembrane regions are bolded, and extracellular regions are italicized of TMIDG2 isoforms 1, 2, and 3.

As disclosed herein, TMIGD2 has been found to be expressed in various human hematologic malignancies and to be functionally important for leukemia-initiating cells and associated with worse overall survival of AML patients. Knock down of TMIGD2 expression was found to impair AML stem cells maintenance and increase cell death of human hematologic malignancies. In some embodiments, treatment of anti-TMIGD2 monoclonal antibodies inhibits AML progress in vivo. Based on these findings, the present disclosure provides methods for treating hematologic malignancies using one or more agents that inhibit TMIGD2 expression and/or activity, as well as agents and kits for use in these methods and the use of these agents and kits to inhibit TMIGD2 expression and/or activity. Exemplary agents for inhibiting TMIGD2 expression and/or activity include, but are not limited to, mRNA targeting agents such as antisense agents or RNAi agents, gene editing agents such as TALEN, ZFN, or CRISPR-based gene editing agents, small molecules, antagonistic antibodies and fusion proteins thereof, and TMIGD2 binding polypeptides.

While the present disclosure is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

### Definitions

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

An agent that “inhibits” TMIGD2 expression and/or activity as used herein reduces TMIGD2 expression and/or activity by at least 5% versus TMIGD2 expression and/or activity in the absence of the agent. In certain embodiments, the agent may reduce TMIGD2 expression and/or activity by at least 10%, at least 20%, at least 30%, at least 40%, at least 50%, at least 60%, at least 70%, at least 80%, at least 90%, at least 95%, at least 99%, or 100% (i.e., complete inhibition) versus TMIGD2 expression and/or activity in the absence of the agent.

The term “antibody” as used herein refers to an immunoglobulin molecule or an immunologically active portion thereof that binds to a specific antigen, e.g., TMIGD2. In those embodiments where an antibody for use in the present methods, compositions, and kits is a full-length immunoglobulin molecule, the antibody comprises two heavy chains and two light chains, with each heavy and light chain containing three complementary determining regions (CDRs). In those embodiments wherein the antibody is an immunologically active portion of an immunoglobulin molecule, the antibody may be, for example, a Fab, Fab′, Fv, Fab′ F(ab′)2, disulfide-linked Fv, scFv, single domain antibody (dAb), or a diabody. Antibodies for use in the present methods, compositions, and kits may include natural antibodies, synthetic antibodies, monoclonal antibodies, polyclonal antibodies, chimeric antibodies, humanized antibodies, multispecific antibodies, bispecific antibodies, dual-specific antibodies, anti-idiotypic antibodies, or fragments thereof that retain the ability to bind a specific antigen, e.g., TMIGD2.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibiting mRNA translation or degrading mRNA molecules. As used herein, the term “RNAi” refers to any type of interfering RNA, including but not limited to siRNAi, shRNAi, endogenous microRNA, and artificial microRNA. For instance, it includes sequences previously identified as siRNA, regardless of the mechanism of downstream processing of the RNA (i.e., although siRNAs are believed to have a specific method of in vivo processing resulting in the cleavage of mRNA, such sequences can be incorporated into the vectors in the context of the flanking sequences described herein).

Ranges recited herein are intended as continuous ranges, including every value between the minimum and maximum values recited, as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein, and in all instances such ratios, ranges, and ranges of ratios represent various embodiments of the present disclosure.

### Methods

Provided herein are methods of treating a condition responsive to TMIGD2 inhibition in a subject in need thereof comprising administering to the subject an agent that inhibits the expression and/or activity of TMIGD2. In certain embodiments, this agent may be an antibody agent, mRNA targeting agent (e.g., antisense agent or RNAi agent), small molecule agent, gene editing agent (e.g., TALEN-based agent, ZFN-based agent, CRISPR-based agent), or polypeptide agent. In certain embodiments, administration of the agent results in an enhanced immune response.

In certain embodiments, the condition responsive to TMIGD2 inhibition is cancer. In some of these embodiments, the cancer is chronic lymphocytic leukemia (CLL), acute leukemia, acute lymphoid leukemia (ALL), B-cell acute lymphoid leukemia (B-ALL), T-cell acute lymphoid leukemia (T-ALL), T-cell lymphoma, B-cell lymphoma, chronic myelogenous leukemia (CML), acute myelogenous leukemia, B-cell prolymphocytic leukemia, blastic plasmacytoid dendritic cell neoplasm, Burkitt's lymphoma, diffuse large B-cell lymphoma, follicular lymphoma, hairy cell leukemia, small cell follicular lymphoma, large cell follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia. In other embodiments, the cancer is a human hematologic malignancy such as myeloid neoplasm, acute myeloid leukemia (AML), AML with recurrent genetic abnormalities, AML with myelodysplasia-related changes, therapy-related AML, acute leukemias of ambiguous lineage, myeloproliferative neoplasm, essential thrombocythemia, polycythemia vera, myelofibrosis (MF), primary myelofibrosis, systemic mastocytosis, myelodysplastic syndromes (MDS), myeloproliferative/myelodysplastic syndromes, chronic myeloid leukemia, chronic neutrophilic leukemia, chronic eosinophilic leukemia, myelodysplastic syndromes (MDS), refractory anemia with ringed sideroblasts, refractory cytopenia with multilineage dysplasia, refractory anemia with excess blasts (type 1), refractory anemia with excess blasts (type 2), MDS with isolated del (5q), unclassifiable MDS, myeloproliferative/myelodysplastic syndromes, chronic myelomonocytic leukemia, atypical chronic myeloid leukemia, juvenile myelomonocytic leukemia, unclassifiable myeloproliferative/myelodysplatic syndromes, lymphoid neoplasms, precursor lymphoid neoplasms, B lymphoblastic leukemia, B lymphoblastic lymphoma, T lymphoblastic leukemia, T lymphoblastic lymphoma, mature B-cell neoplasms, diffuse large B-cell lymphoma, primary central nervous system lymphoma, primary mediastinal B-cell lymphoma, Burkitt lymphoma/leukemia, follicular lymphoma, chronic lymphocytic leukemia, small lymphocytic lymphoma, B-cell prolymphocytic leukemia, lymphoplasmacytic lymphoma, Waldenstrom macroglobulinemia, mantle cell lymphoma, marginal zone lymphomas, post-transplant lymphoproliferative disorders, HIV-associated lymphomas, primary effusion lymphoma, intravascular large B-cell lymphoma, primary cutaneous B-cell lymphoma, hairy cell leukemia, multiple myeloma, monoclonal gammopathy of unknown significance (MGUS), smoldering multiple myeloma, or solitary plasmacytomas (solitary bone and extramedullary).

In some embodiments, the cancer is Adrenal Cancer, Anal Cancer, Basal and Squamous Cell Skin Cancer, Bile Duct Cancer, Bladder Cancer, Bone Cancer, Brain and Spinal Cord Tumors, Breast Cancer, Cervical Cancer, Colorectal Cancer, Endometrial Cancer, Esophagus Cancer, Ewing Family of Tumors, Eye Cancer (Ocular Melanoma), Gallbladder Cancer, Gastrointestinal Neuroendocrine (Carcinoid) Tumors, Gastrointestinal Stromal Tumor (GIST), Gestational Trophoblastic Disease, Kaposi Sarcoma, Kidney Cancer, Laryngeal and Hypopharyngeal Cancer, Liver Cancer, Lung Cancer, Lung Carcinoid Tumor, Malignant Mesothelioma, Melanoma Skin Cancer, Merkel Cell Skin Cancer, Nasal Cavity and Paranasal Sinuses Cancer, Nasopharyngeal Cancer, Neuroblastoma, Non-Small Cell Lung Cancer, neoplasm of the central nervous system (CNS), Oral Cavity and Oropharyngeal Cancer, Osteosarcoma, Ovarian Cancer, Pancreatic Cancer, Pancreatic Neuroendocrine Tumor (NET), Penile Cancer, Pituitary Tumors, Prostate Cancer, Retinoblastoma, Rhabdomyosarcoma, Salivary Gland Cancer, Skin Cancer, Small Cell Lung Cancer, Small Intestine Cancer, Soft Tissue Sarcoma, Stomach Cancer, Testicular Cancer, Thymus Cancer, Thyroid Cancer, Uterine Sarcoma, Vaginal Cancer, Vulvar Cancer, Waldenstrom Macroglobulinemia, Wilms Tumor, squamous cell cancer, environmentally induced cancers, combinations of the cancers, and metastatic lesions of the cancers. In some embodiments, the cancer is leukemia or lymphoma, for example, lymphoblastic lymphoma or B-cell Non-Hodgkin's lymphoma.

In certain embodiments, the methods provided herein further comprise administering a second agent. In certain of these embodiments, the second agent also inhibits TMIGD2 expression and/or activity. In other embodiments, the second agent is a non-TMIGD2 inhibiting agent used in the treatment of the condition, including for example radiation treatment or chemotherapy. In these combination embodiments, the first and second agents may be administered simultaneously or sequentially, via the same or different routes. When the agents are administered simultaneously, they may be administered in a single formulation or in separate formulations. When the agents are administered sequentially, they may be administered at the same or different intervals. For example, one agent may be administered more frequently than the other, or may be administered over a longer time course. In certain embodiments, the second agent may be administered one or more hours, days, or weeks after the first agent, or vice versa. In certain embodiments, one agent may be administered one or more times prior to the first administration of the second agent. When administration of the second agent is initiated, administration of the first agent may either cease or continue for all or part of the course of administration of the second agent.

### A. Antibody Agents

In certain embodiments of the methods provided herein, the agent for inhibiting TMIGD2 expression and/or activity is an antibody or antigen-binding fragment thereof or a fusion protein thereof.

In these embodiments, the antibody or antigen-binding fragment thereof specifically binds an epitope of TMIGD2, e.g., TMIGD2 isoform 1, 2, or 3 as set forth in SEQ ID NOs: 1-3, respectively. In certain embodiments, the antibody or antigen-binding fragment thereof is cross-reactive with two or more TMIGD2 isoforms, while in other embodiments the antibody is specific to a single isoform. For example, in certain embodiments the antibody or antigen-binding fragment thereof may bind both isoforms 1 and 2 but not isoform 3, or vice versa. In some embodiments, the antibodies or antigen-binding fragments thereof bind human TMIGD2 only. In other embodiments, the antibodies or antigen-binding fragments thereof bind non-human TMIGD2 (e.g., mouse TMIGD2) in addition to or in lieu of human TMIGD2. In some embodiments, the antibodies or antigen-binding fragments thereof partially or completely block binding of TMIGD2 to HHLA. In certain embodiments, the antibody or antigen-binding fragment thereof modulates (e.g., inhibits) one or more aspects of TMIGD2 signaling (such as TMIDG2 phosphorylation).

In certain embodiments, the antibody or antigen-binding fragment thereof binds an epitope located completely or partially in the extracellular domain of TMIGD2, e.g., residues 1-150 of SEQ ID NOs: 1 or 2 or residues 1-30 of SEQ ID NO:3. In some of these embodiments, the antibody or antigen-binding fragment thereof binds the extracellular domain of all TMIGD2 isoforms. In other embodiments, the antibody or antigen-binding fragment thereof is specific to one or more isoforms. For example, the antibody or antigen-binding fragment thereof may bind the extracellular domain of isoforms 1 and 2 but not isoform 3, or vice versa.

In some embodiments, the antibody or antigen-binding fragment thereof is a one-armed antibody (i.e., the heavy chain variable domain and the light chain variable domain form a single antigen binding arm) comprising an Fc region, wherein the Fc region comprises a first and a second Fc polypeptide, wherein the first and second Fc polypeptides are present in a complex and form a Fc region that increases stability of said antibody fragment compared to a Fab molecule comprising said antigen binding arm.

In one embodiment, the antibody or antigen-binding fragment thereof is a chimeric antibody, for example, an antibody comprising antigen binding sequences from a non-human donor grafted to a heterologous non-human, human, or humanized sequence (e.g., framework and/or constant domain sequences). In one embodiment, the non-human donor is a mouse. In a further embodiment, an antigen binding sequence is synthetic, e.g., obtained by mutagenesis (e.g., phage display screening, etc.). In a particular embodiment, a chimeric antibody of the invention has murine V regions and a human C region. In one embodiment, the murine light chain V region is fused to a human kappa light chain. In another embodiment, the murine heavy chain V region is fused to a human IgG1 C region.

In some embodiments, an antibody or antigen-binding fragment thereof described herein is or comprises: (i) a chimeric antibody, a human antibody, or a humanized antibody, or antigen-binding fragment thereof; (ii) a monospecific antibody or a bispecific antibody, or antigen-binding fragment thereof; and/or (iii) a monoclonal antibody, or antigen-binding fragment thereof. In some embodiments, an antibody or antigen-binding fragment thereof described herein can be or comprise an immunoglobulin, heavy chain antibody, light chain antibody, or other protein scaffold with antibody-like properties, as well as other immunological binding moieties known in the art, including, but not limited to, a Fab fragment, a Fab′ fragment, a F(ab′) 2 fragment, a Fv fragment, a disulfide-bonded Fv fragment, a scFv fragment, a diabody, a triabody, a tetrabody, a minibody, a maxibody, a tandab, a BiTe, a nanobody, a camelid antibody, or any combination thereof. In some embodiments, an antibody or antigen-binding fragment thereof is or comprises: (i) a heavy chain constant region chosen from IgG1, IgG2, IgG3, or IgG4, and/or (ii) a light chain constant region chosen from the light chain constant regions of kappa or lambda.

In some embodiments, an antibody or antigen-binding fragment thereof is or comprises: (a) a heavy chain variable region (VH) comprising one, two, or three VH CDR sequences each with at least about 90% identity to a VH CDR of Table 3 or 4; and/or (b) a light chain variable region (VL) comprising one, two, or three VL CDR sequences each with at least about 90% identity to a VL CDR of Table 3 or 4. In some embodiments, an antibody or antigen-binding fragment thereof is or comprises: (a) a VH comprising one, two, or three VH CDR sequences each with at least about 91%, 92%, 93%, 94%, 95%, 96%, 97%, 98%, 99%, 99.5% or higher identity to a VH CDR of Table 3 or 4; and/or (b) a VL comprising one, two, or three VL CDR sequences each with at least about 91%, 92%, 93%, 94%, 95%, 96%, 97%, 98%, 99%, 99.5% or higher identity to a VL CDR of Table 3 or 4. In some embodiments, an antibody or antigen-binding fragment thereof is or comprises: (a) a VH comprising one, two, or three VH CDR sequences each comprising or consisting of a VH CDR of Table 3 or 4; and/or (b) a VL comprising one, two, or three VL CDR sequences each comprising or consisting of a VL CDR of Table 3 or 4.

In some embodiments, an antibody or antigen-binding fragment thereof is or comprises: (a) a VH with at least about 90% or more identity to a VH of Table 3 or 4; and/or (b) a VL with at least about 90% or more identity to a VL of Table 3 or 4. In some embodiments, an antibody or antigen-binding fragment thereof is or comprises: (a) a VH with at least about 95%, 96%, 97%, 98%, 99%, 99.5% or higher identity to a VH of Table 3 or 4; and/or (b) a VL with at least about 95%, 96%, 97%, 98%, 99%, 99.5% or higher identity to a VL of Table 3 or 4. In some embodiments, an antibody or antigen-binding fragment thereof is or comprises: (a) a VH comprising or consisting of a VH of Table 3 or 4; and/or (b) a VL comprising or consisting of a VL of Table 3 or 4.

In some embodiments, an antibody or antigen-binding fragment thereof is or comprises: (a) a heavy chain with at least about 90% or more identity to a heavy chain of Table 3 or 4; and/or (b) a light chain with at least about 90% or more identity to a light chain of Table 3 or 4. In some embodiments, an antibody or antigen-binding fragment thereof is or comprises: (a) a heavy chain with at least about 95%, 96%, 97%, 98%, 99%, 99.5% or higher identity to a heavy chain of Table 3 or 4; and/or (b) a light chain with at least about 95%, 96%, 97%, 98%, 99%, 99.5% or higher identity to a light chain of Table 3 or 4. In some embodiments, an antibody or antigen-binding fragment thereof is or comprises: (a) a heavy chain comprising or consisting of a heavy chain of Table 3 or 4; and/or (b) a light chain comprising or consisting of a light chain of Table 3 or 4.

In certain embodiments, an antibody or antigen-binding fragment thereof described herein is conjugated to a cytotoxic agent. In such embodiments, a cytotoxic agent is selected from the group consisting of a therapeutic agent (e.g., a chemotherapeutic agent), a biologic agent, a toxin, and a radioactive isotope. Exemplary cytotoxic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, or 5-fluorouracil decarbazine), alkylating agents (e.g, mechlorethamine, thioepachlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g, dactinomycin (formerly actinomycin), bleomycin, mithramycin, and anthramycin (AMC)), and anti-mitotic agents (e.g., vincristine and vinblastine). An antibody or antigen-binding fragment thereof described herein can be conjugated to a radioisotope, e.g, radioactive iodine, to generate cytotoxic radiopharmaceuticals for treating a related disorder, such as a cancer described herein.

Antibody conjugates can be used to modify a given biological response. A therapeutic moiety is not to be construed as limited to classical chemical therapeutic agents. For example, a drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-. gamma.; or, biological response modifiers such as, for example, lymphokines, interleukin-1 (“IL-I”), interleukin-2 (“IL-2”), interleukin-6 (“IL-6”), granulocyte macrophage colony stimulating factor (“GM-CSF”), granulocyte colony stimulating factor (“G-CSF”), or other cytokines or growth factors. Techniques for conjugating such therapeutic moiety to antibodies are well-known, see, e.g., Arnon et al, “Monoclonal Antibodies For Immunotargeting Of Drugs In Cancer Therapy”, in Monoclonal Antibodies And Cancer Therapy, Reisfeld et al. (eds.), pp. 243 56 (Alan R. Liss, Inc. 1985); Hellstrom et al, “Antibodies For Drug Delivery”, in Controlled Drug Delivery (2nd Ed.), Robinson et al. (eds.), pp. 623 53 (Marcel Dekker, Inc. 1987); Thorpe, “Antibody Carriers Of Cytotoxic Agents In Cancer Therapy: A Review”, in Monoclonal Antibodies ‘84: Biological And Clinical Applications, Pinchera et al. (eds.), pp. 475 506 (1985); “Analysis, Results, And Future Prospective Of The Therapeutic Else Of Radiolabeled Antibody In Cancer Therapy”, in Monoclonal Antibodies For Cancer Detection And Therapy, Baldwin et al. (eds.), pp. 303 16 (Academic Press 1985), and Thorpe et al., “The Preparation And Cytotoxic Properties Of Antibody-Toxin Conjugates”, Immunol. Rev., 62:119 58 (1982).

In some embodiments, conjugations can be made using a “cleavable linker” facilitating release of the cytotoxic agent or growth inhibitory agent in a cell. For example, an acid-labile linker, peptidase-sensitive linker, photolabile linker, dimethyl linker or disulfide-containing linker (See e.g. U.S. Pat. No. 5,208,020) may be used. Alternatively, a fusion protein comprising an antibody or antigen-binding fragment thereof and cytotoxic agent or growth inhibitory agent may be made, by recombinant techniques or peptide synthesis. The length of DNA may comprise respective regions encoding the two portions of the conjugate either adjacent one another or separated by a region encoding a linker peptide which does not destroy the desired properties of the conjugate.

In some embodiments, an antibody or antigen-binding fragment thereof is a 17C7 monoclonal antibody clone, or comprises heavy and/or light chain sequences, heavy and/or light chain variable sequences, or one or more CDR sequences of the 17C7 monoclonal antibody clone. The sequences of the 17C7 antibody are disclosed in Table 3 below:

TABLE 3
17C7 Antibody Sequences

| | DNA Sequence | Amino Acid Sequence |
|---|---|---|
| VH | GAGGTTCAGCTGCAGCAGTCT | EVQLQQSGPELVKPGTLVNISCKA |
| | GGACCTGAGCTGGTGAAGCCT | SGYTFTSYDINWVKQRPGQGLEWI |
| | GGGACTTTAGTGAATATATCCT | GWIYPGDGSTNYNEKFKGKATLTA |
| | GCAAGGCTTCTGGTTACACCTT | DKSSSTAYMQLSSLTSENSAVYFC |
| | CACAAGCTACGATATAAACTGG | ARRGLRYYFDYWGQGTTLTVSS |
| | GTGAAGCAGAGGCCTGGACAG | (SEQ ID NO: 23) |
| | GGACTTGAGTGGATTGGATGGA | |
| | TTTATCCTGGAGATGGTAGTAC | |
| | TAACTACAATGAGAAATTCAAG | |
| | GGCAAGGCCACACTGACTGCA | |
| | GACAAATCCTCCAGCACAGCCT | |
| | ACATGCAGCTCAGCAGCCTGAC | |
| | TTCTGAGAACTCTGCAGTCTAT | |
| | TTCTGTGCAAGAAGAGGGCTAC | |
| | GGTACTACTTTGACTACTGGGG | |
| | CCAAGGCACCACTCTCACAGTC | |
| | TCCTCA (SEQ ID NO: 7) | |
| VH-CDR1 | GGTTACACCTTCACAAGCTACG | GYTFTSYDIN (SEQ ID NO: 24) |
| | ATATAAAC (SEQ ID NO: 8) | |
| VH-CDR2 | TGGATTTATCCTGGAGATGGTA | WIYPGDGSTNYNEKFKG (SEQ ID |
| | GTACTAACTACAATGAGAAATT | NO: 25) |
| | CAAGGGC (SEQ ID NO: 9) | |
| VH-CDR3 | GCAAGAAGAGGGCTACGGTAC | ARRGLRYYFDY (SEQ ID NO: 26) |
| | TACTTTGACTAC (SEQ ID NO: 10) | |
| VH-FR1 | GAGGTTCAGCTGCAGCAGTCT | EVQLQQSGPELVKPGTLVNISCKA |
| | GGACCTGAGCTGGTGAAGCCT | S (SEQ ID NO: 27) |
| | GGGACTTTAGTGAATATATCCT | |
| | GCAAGGCTTCT (SEQ ID NO: 11) | |
| VH-FR2 | TGGGTGAAGCAGAGGCCTGGA | WVKQRPGQGLEWIG (SEQ ID NO: |
| | CAGGGACTTGAGTGGATTGGA | 28) |
| | (SEQ ID NO: 12) | |
| VH-FR3 | AAGGCCACACTGACTGCAGACA | KATLTADKSSSTAYMQLSSLTSEN |
| | AATCCTCCAGCACAGCCTACAT | SAVYFC (SEQ ID NO: 29) |
| | GCAGCTCAGCAGCCTGACTTCT | |
| | GAGAACTCTGCAGTCTATTTCT | |
| | GT (SEQ ID NO: 13) | |
| VH-FR4 | TGGGGCCAAGGCACCACTCTC | WGQGTTLTVSS (SEQ ID NO: 30) |
| | ACAGTCTCCTCA (SEQ ID NO: | |
| | 14) | |
| VL | GATATCCAGATGACACAGACTA | DIQMTQTTSSLSASLGDRVTISCRA |
| | CATCCTCCCTGTCTGCCTCTCT | SQDIRNYLNWYQQKPDGTVKLLIY |
| | GGGAGACAGAGTCACCATTAGT | YTSRLHSGVPSRFSGSGSGTDYSL |
| | TGCAGGGCAAGTCAGGACATTA | TISNLEQEDIATYFCQQVNTLPWTF |
| | GGAATTATTTAAACTGGTATCA | GGGTKLEIK (SEQ ID NO: 31) |
| | GCAGAAACCAGATGGCACTGTT | |
| | AAACTCCTGATCTACTACACAT | |
| | CAAGATTACATTCAGGAGTCCC | |
| | ATCAAGGTTCAGTGGCAGTGG | |
| | GTCTGGAACAGATTATTCTCTC | |
| | ACCATTAGCAACCTGGAGCAAG | |
| | AAGATATTGCCACTTACTTTTGC | |
| | CAACAGGTTAATACGCTTCCGT | |
| | GGACGTTCGGTGGAGGCACCA | |
| | AGCTGGAAATCAAA (SEQ ID NO: | |
| | 15) | |
| VL-CDR1 | AGGGCAAGTCAGGACATTAGG | RASQDIRNYLN (SEQ ID NO: 32) |
| | AATTATTTAAAC (SEQ ID NO: 16) | |
| VL-CDR2 | TACACATCAAGATTACATTCA | YTSRLHS (SEQ ID NO: 33) |
| | (SEQ ID NO: 17) | |
| VL-CDR3 | CAACAGGTTAATACGCTTCCGT | QQVNTLPWT (SEQ ID NO: 34) |
| | GGACG (SEQ ID NO: 18) | |
| VL-FR1 | GATATCCAGATGACACAGACTA | DIQMTQTTSSLSASLGDRVTISC |
| | CATCCTCCCTGTCTGCCTCTCT | (SEQ ID NO: 35) |
| | GGGAGACAGAGTCACCATTAGT | |
| | TGC (SEQ ID NO: 19) | |
| VL-FR2 | TGGTATCAGCAGAAACCAGATG | WYQQKPDGTVKLLIY (SEQ ID NO: |
| | GCACTGTTAAACTCCTGATCTA | 36) |
| | C (SEQ ID NO: 20) | |
| VL-FR3 | GGAGTCCCATCAAGGTTCAGTG | GVPSRFSGSGSGTDYSLTISNLEQ |
| | GCAGTGGGTCTGGAACAGATTA | EDIATYFC (SEQ ID NO: 37) |
| | TTCTCTCACCATTAGCAACCTG | |
| | GAGCAAGAAGATATTGCCACTT | |
| | ACTTTTGC (SEQ ID NO: 21) | |
| VL-FR4 | TTCGGTGGAGGCACCAAGCTG | FGGGTKLEIK (SEQ ID NO: 38) |
| | GAAATCAAA (SEQ ID NO: 22) | |

In some embodiments, an antibody or antigen-binding fragment thereof is a 20F2 monoclonal antibody clone, or comprises heavy and/or light chain sequences, heavy and/or light chain variable sequences, or one or more CDR sequences of the 20F2 monoclonal antibody clone. The sequences of the 20F2 antibody are disclosed in Table 4 below:

TABLE 4
20F2 Antibody Sequences

| | DNA Sequence | Amino Acid Sequence |
|---|---|---|
| VH | CAGCTGAAGCAGTCAGGACCT | QLKQSGPGLVKPSQSLSLTCTVTG |
| | GGCCTGGTGAAACCTTCTCAGT | YSITSDYAWNWIRQFPGNKLELMG |
| | CACTGTCCCTCACCTGCACTGT | YITYSGSTSYNPSLKSRFSITRDTS |
| | CACTGGCTACTCAATCACCAGT | KNQFFLQLNSVTTEDTATYYCARS |
| | GATTATGCCTGGAACTGGATCC | GYRYDDAMDYWGQGTSVTVSS |
| | GGCAGTTTCCAGGAAACAAACT | (SEQ ID NO: 55) |
| | GGAGTTGATGGGCTACATAACC | |
| | TACAGTGGTAGCACTAGCTACA | |
| | ACCCATCTCTCAAAAGTCGATT | |
| | CTCTATCACTCGAGACACATCC | |
| | AAGAACCAGTTCTTCCTGCAGT | |
| | TGAATTCTGTGACTACTGAGGA | |
| | CACAGCCACATATTACTGTGCA | |
| | AGATCGGGGTATAGGTACGAC | |
| | GATGCTATGGACTACTGGGGTC | |
| | AAGGAACCTCAGTCACCGTCTC | |
| | CTCA (SEQ ID NO: 39) | |
| VH-CDR1 | GGCTACTCAATCACCAGTGATT | GYSITSDYAWN (SEQ ID NO: 56) |
| | ATGCCTGGAAC (SEQ ID NO: 40) | |
| VH-CDR2 | TACATAACCTACAGTGGTAGCA | YITYSGSTSYNPSLKS (SEQ ID NO: |
| | CTAGCTACAACCCATCTCTCAA | 57) |
| | AAGT (SEQ ID NO: 41) | |
| VH-CDR3 | GCAAGATCGGGGTATAGGTAC | ARSGYRYDDAMDY (SEQ ID NO: |
| | GACGATGCTATGGACTAC (SEQ | 58) |
| | ID NO: 42) | |
| VH-FR1 | CAGCTGAAGCAGTCAGGACCT | QLKQSGPGLVKPSQSLSLTCTVT |
| | GGCCTGGTGAAACCTTCTCAGT | (SEQ ID NO: 59) |
| | CACTGTCCCTCACCTGCACTGT | |
| | CACT (SEQ ID NO: 43) | |
| VH-FR2 | TGGATCCGGCAGTTTCCAGGAA | WIRQFPGNKLELMG (SEQ ID NO: |
| | ACAAACTGGAGTTGATGGGC | 60) |
| | (SEQ ID NO: 44) | |
| VH-FR3 | CGATTCTCTATCACTCGAGACA | RFSITRDTSKNQFFLQLNSVTTEDT |
| | CATCCAAGAACCAGTTCTTCCT | ATYYC (SEQ ID NO: 61) |
| | GCAGTTGAATTCTGTGACTACT | |
| | GAGGACACAGCCACATATTACT | |
| | GT (SEQ ID NO: 45) | |
| VH-FR4 | TGGGGTCAAGGAACCTCAGTCA | WGQGTSVTVSS (SEQ ID NO: 62) |
| | CCGTCTCCTCA (SEQ ID NO: 46) | |
| VL | GACATTGTGATGACACAGTCTC | DIVMTQSPSSLAMSVGQKVTMSCK |
| | CATCCTCCCTGGCTATGTCAGT | SSQSLLSSNNQKNYLAWYQLKPG |
| | CGGACAGAAGGTCACTATGAG | QSPKLLVYFASTRESGVPDRFIGS |
| | CTGCAAGTCCAGTCAGAGCCTT | GSGTDFTLTISSVQAEDLADYFCQ |
| | TTAAGTAGTAATAATCAAAAGAA | QHYRTPLTFGAGTKLELK (SEQ ID |
| | CTATTTGGCCTGGTACCAGCTG | NO: 63) |
| | AAACCAGGACAGTCTCCTAAAC | |
| | TTCTGGTATATTTTGCATCCACT | |
| | AGGGAATCTGGGGTCCCTGAT | |
| | CGCTTCATAGGCAGTGGATCTG | |
| | GGACAGATTTCACTCTTACTAT | |
| | CAGCAGTGTGCAGGCTGAAGA | |
| | CCTGGCAGATTACTTCTGTCAG | |
| | CAACATTATCGCACTCCGCTCA | |
| | CGTTCGGTGCTGGGACCAAGC | |
| | TGGAGCTGAAA (SEQ ID NO: 47) | |
| VL-CDR1 | AAGTCCAGTCAGAGCCTTTTAA | KSSQSLLSSNNQKNYLA (SEQ ID |
| | GTAGTAATAATCAAAAGAACTAT | NO: 64) |
| | TTGGCC (SEQ ID NO: 48) | |
| VL-CDR2 | TTTGCATCCACTAGGGAATCT | FASTRES (SEQ ID NO: 65) |
| | (SEQ ID NO: 49) | |
| VL-CDR3 | CAGCAACATTATCGCACTCCGC | QQHYRTPLT (SEQ ID NO: 66) |
| | TCACG (SEQ ID NO: 50) | |
| VL-FR1 | GACATTGTGATGACACAGTCTC | DIVMTQSPSSLAMSVGQKVTMSC |
| | CATCCTCCCTGGCTATGTCAGT | (SEQ ID NO: 67) |
| | CGGACAGAAGGTCACTATGAG | |
| | CTGC (SEQ ID NO: 51) | |
| VL-FR2 | TGGTACCAGCTGAAACCAGGAC | WYQLKPGQSPKLLVY (SEQ ID NO: |
| | AGTCTCCTAAACTTCTGGTATAT | 68) |
| | (SEQ ID NO: 52) | |
| VL-FR3 | GGGGTCCCTGATCGCTTCATAG | GVPDRFIGSGSGTDFTLTISSVQAE |
| | GCAGTGGATCTGGGACAGATTT | DLADYFC (SEQ ID NO: 69) |
| | CACTCTTACTATCAGCAGTGTG | |
| | CAGGCTGAAGACCTGGCAGATT | |
| | ACTTCTGT (SEQ ID NO: 53) | |
| VL-FR4 | TTCGGTGCTGGGACCAAGCTG | FGAGTKLELK (SEQ ID NO: 70) |
| | GAGCTGAAA (SEQ ID NO: 54) | |

B. mRNA Targeting Agents

In certain embodiments of the methods provided herein, the agent for inhibiting TMIGD2 expression and/or activity is an mRNA targeting agent such as an antisense agent or RNAi agent.

In certain embodiments of the methods provided herein, the mRNA targeting agent is an antisense agent. Antisense agents, typically small fragments of DNA or RNA, modulate protein expression by binding to a target mRNA encoding the protein, forming a hybrid duplex. Intracellularly, the antisense agent/mRNA hybrid is cleaved by ribonuclease H (RNAse H). RNAse H-mediated cleavage of the RNA strand from the duplex results in the mRNA being unable to be translated to the protein.

In certain embodiments of the methods provided herein, the mRNA targeting agent is an RNAi agent. RNA interference (RNAi) is an evolutionarily conserved process whereby the expression or introduction of RNA of a sequence that is identical or highly similar to a target gene results in sequence specific degradation or specific post-transcriptional gene silencing (PTGS) of messenger RNA (mRNA) transcribed from that targeted gene, thereby inhibiting expression of the target gene. RNAi agents are typically comprised of a sequence of nucleic acids or nucleic acid analogs specific for a target gene (e.g., TMIGD2). In some embodiments, the RNAi agent comprises or is a small nucleic acid molecule such as a short interfering RNA (siRNA), double-stranded RNA (dsRNA), micro-RNA (miRNA), piwi-RNA (piRNA), small nucleolar RNA (snoRNA), tRNA-derived small RNAs (tsRNAs), small regulatory RNA (srRNA), or short hairpin RNA (shRNA) molecule. An siRNA agent refers to a nucleic acid that forms a double stranded RNA having the ability to reduce or inhibit expression of a TMIGD2 gene when the siRNA is present or expressed in the same cell as the TMIGD2 gene. shRNA is a type of siRNA that functions similar to RNAi and/or siRNA species, but differs in that shRNA species have double stranded hairpin-like structure for increased stability. In some embodiments, an shRNA agent reduces or inhibits expression of a TMIGD2 gene when the shRNA is present or expressed in the same cells as the TMIGD2 gene. In a further of such embodiment, an shRNA increases expression of genes involved in apoptosis and cell cycle arrest. miRNA are endogenous RNAs, some of which are known to regulate the expression of protein-coding genes at the posttranscriptional level. Endogenous microRNA are small RNAs naturally present in the genome which are capable of modulating the productive utilization of mRNA. In some embodiments, the miRNA agent is an artificial miRNA agent, which includes any type of RNA sequence, other than endogenous microRNA, which is capable of modulating the productive utilization of mRNA. A dsRNA agent is an RNA molecule comprised of two strands. The dsRNA agents include RNA molecules comprised of a single RNA molecule that doubles back on itself to form a two-stranded structure. For example, the stem loop structure of the progenitor molecules from which the single-stranded miRNA is derived, called the pre-miRNA, comprises a dsRNA molecule. A piRNA agent refers to a nucleic acid molecule that forms RNA-protein complexes through interactions with piwi-subfamily Argonaute proteins and having the ability to reduce or inhibit expression of a TMIGD2 gene when the piRNA is present or expressed in the same cell as the TMIGD2 gene. A snoRNA agent refers to a nucleic acid that guides chemical modifications of other RNAs having the ability to reduce or inhibit expression of a TMIGD2 gene when the snoRNA is present or expressed in the same cell as the TMIGD2 gene.

In some embodiments, an mRNA targeting agent for use in the methods provided herein comprises or consists of a nucleic acid sequence complementary to all or a portion of a TMIGD2 mRNA sequence. For example, in certain embodiments the mRNA targeting agent comprises or consists of a nucleic acid sequence complementary to all or a portion of a TMIGD2 mRNA encoded by SEQ ID NO:4 (exemplary DNA sequence of TMIGD2 isoform 1), SEQ ID NO:5 (exemplary DNA sequence of TMIGD2 isoform 2), or SEQ ID NO: 6 (exemplary DNA sequence of TMIGD2 isoform 3). In certain of these embodiments, the mRNA targeting agent may be complementary to a specific region of a TMIGD2 mRNA. For example, in certain embodiments the mRNA targeting agent is complementary to a portion of TMIGD2 mRNA corresponding to the extracellular domain of TMIGD2, e.g., an mRNA corresponding to residues 1-150 of SEQ ID NOs: 1 or 2 or residues 1-30 of SEQ ID NO: 3, including an mRNA encoded by nucleotides 1-450 of SEQ ID NOs: 4 or 5 or nucleotides 1-90 of SEQ ID NO:6; a portion of TMIGD2 mRNA corresponding to the transmembrane domain of TMIGD2, e.g., an mRNA corresponding to residues 151-171 of SEQ ID NOs: 1 or 2 or residues 31-51 of SEQ ID NO:3, including an mRNA encoded by nucleotides 451-513 of SEQ ID NOs: 4 or 5 or nucleotides 91-153 of SEQ ID NO:6; or a portion of TMIGD2 mRNA corresponding to the intracellular domain of TMIGD2, e.g., an mRNA corresponding to residues 172-282 of SEQ ID NO:1, residues 172-278 of SEQ ID NO: 2, or residues 52-162 of SEQ ID NO:3, including an mRNA encoded by nucleotides 514-849 of SEQ ID NO:4, nucleotides 514-837 of SEQ ID NO:5, or nucleotides 154-489 of SEQ ID NO: 6.

It is understood in the art that a nucleic acid molecule need not be 100% complementary to a target nucleic acid sequence in order to specifically hybridize to the target sequence. Accordingly, in certain embodiments an mRNA targeting agent for use in the methods provided herein may be at least about 50%, at least about 60%, at least about 70%, at least about 80%, at least about 90%, at least about 95%, at least about 99%, or 100% complementary to all or a portion of the TMIGD2 mRNA target sequence.

In certain embodiments, an mRNA targeting agent for use in the methods provided herein may be essentially fully complementary to all or a portion of a TMIGD2 mRNA sequence. An mRNA targeting agent of the present disclosure and a TMIGD2 mRNA are essentially fully complementary to one another when the degree of complementarity permits stable and specific binding between the mRNA targeting agent and the TMIGD2 mRNA. In certain embodiments, mRNA targeting agents having one or two non-complementary nucleobases with respect to a TMIGD2 mRNA may be considered essentially fully complementary.

In certain embodiments, an mRNA targeting agent of the present disclosure and a target nucleic acid of TMIGD2 are fully complementary to each other. An mRNA targeting agent and a target nucleic acid of TMIGD2 are fully complementary to each other when each nucleobase of the mRNA targeting agent is complementary to an equal number of nucleobases at corresponding positions in the target nucleic acid.

In some embodiments, an mRNA targeting agent for use in the disclosed methods comprises a backbone of linked monomeric subunits where each linked monomeric subunit is directly or indirectly attached to a heterocyclic base moiety. The linkages joining the monomeric subunits, the sugar moieties or sugar surrogates, and the heterocyclic base moieties can be independently modified giving rise to a plurality of motifs for the resulting antisense agents including hemimers, gapmers, alternating, uniformly modified, and positionally modified.

In one embodiment, the mRNA targeting agents of the present disclosure are 10 to 30 nucleosides in length, for example, 15 to 30 linked or contiguous nucleosides, 10 to 25 linked or contiguous nucleosides, 20 to 30 linked or contiguous nucleosides, or 15 to 20 linked or contiguous nucleosides.

Delivery of mRNA targeting agents may be accomplished by any suitable method known in the art, including but not limited to viral vector-delivery (e.g., lentiviral vector delivery, AAV-viral vector delivery, adenoviral vector delivery), dendrimer-mediated delivery, nanoparticle-mediated delivery or a combination thereof (e.g., dendrimer-based nanoparticle delivery).

### C. Small Molecule Agents

In certain embodiments of the methods provided herein, the agent for inhibiting TMIGD2 expression and/or activity is a small molecule agent.

Small molecule cancer drugs have been successfully used to target the extracellular, cell surface ligand-binding receptors as well as the intracellular proteins, including anti-apoptotic proteins that play a key role in transducing downstream signaling for cell growth and metastasis promotion.

In some embodiments, the small molecule agent interferes with TMIGD2 activity by binding to and inhibiting the activity of a TMIGD2 protein, e.g., TMIGD2 isoform 1, 2, and/or 3. In certain of these embodiments, the small molecule binds to the extracellular domain of TMIGD2, e.g., residues 1-150 of SEQ ID NOs: 1 or 2 or residues 1-30 of SEQ ID NO: 3. In some embodiments, the small molecule agent reduces dimerization and/or aggregation of TMIGD2. In some embodiments, the small molecule agent partially or completely blocks TMIGD2 binding to HHLA.

In some embodiments, the small molecule agent interferes with TMIGD2 expression and/or activity by binding to and inhibiting a protein involved in TMIGD2 expression, e.g., an upstream effector of TMIGD2 or a protein or a transcription factor involved in TMIGD2 expression. In other embodiments, the small molecule agent interferes with TMIGD2 expression and/or activity by binding to a TMIGD2 nucleic acid, e.g., a TMIGD2 DNA or mRNA sequence.

### D. Gene Editing Agents

In certain embodiments of the methods provided herein, the agent for inhibiting TMIGD2 expression and/or activity is a gene editing agent.

Gene editing agents include agents comprising one or more DNA or RNA sequences. In certain embodiments, the gene editing agents comprise multiple components. For example, the gene editing agent may comprise multiple vectors encoding different components, e.g., one or more gRNA sequences and one or more nucleases or nucleic acid sequences encoding nucleases.

In certain embodiments, the gene editing agents inhibit TMIGD2 expression and/or activity by altering the TMIGD2 gene sequence or the sequence of a regulatory element associated with the TMIGD2 gene, e.g., a promoter or enhancer element. For example, the gene editing agent may knock out or knock down TMIGD2 expression by introducing a deletion, insertion, or mutation into the TMIGD2 gene or a regulatory element thereof. A gene is considered knocked out upon complete removal of the gene or complete deactivation or suppression of the gene through genetic engineering. A gene is considered knocked down when the gene is partially deactivated or suppressed.

In some embodiments, the gene editing agent may introduce an alteration that entirely prevents TMIGD2 expression, for example by disrupting the start codon of the TMIGD2 gene or a critical regulatory element thereof. In other embodiments, the alteration may result in decreased TMIGD2 expression, or in expression of a truncated, inactive, or partially inactive form of TMIGD2, for example by introducing a nonsense mutation into the gene, disrupting one or more exon sequences of the gene, and/or altering one or more nucleotides encoding a functional domain or element of TMIGD2. In some embodiments, the agent introduces an inactivating mutation into the TMIGD2 gene. In some embodiments, the agent represses transcription of the TMIGD2 gene. In some embodiments, the alteration decreases TMIGD2 expression or activity by at least 10%, at least 20%, at least 30%, at least 40%, at least 50%, at least 60%, at least 70%, at least 80%, at least 90%, at least 95%, at least 99%, or 100% versus expression or activity in the absence of the alteration. In some embodiments, the gene editing agent deletes or alters one or more nucleotides located in the region encoding the extracellular domain, transmembrane domain, or intracellular domain of TMIGD2.

In some embodiments, the agent comprises a programmable nuclease. In some embodiments, the agent comprises a natural homing meganuclease. In some embodiments, the agent is a TALEN-based agent, a ZFN-based agent, or a CRISPR-based agent, or any biologically active fragment, fusion, derivative or combination thereof. In some embodiments, the agent is a deaminase or a nucleic acid encoding a deaminase. In some embodiments, a cell is engineered to stably and/or transiently express a TALEN-based agent, a ZFN-based agent, and/or a CRISPR-based agent.

### I. TALEN-Based Agents

In some embodiments, the gene editing agent is a TALEN-based agent. In some embodiments, the TALEN-based agent is one or more TALEN polypeptides/proteins or biologically active fragments or derivatives thereof, or one more nucleic acids encoding one or more TALEN polypeptides or fragments or derivatives thereof. Transcription activator-like (TAL) effector sequences can be assembled to bind DNA targets with specificity by assembling sequences of repeat variable-diresidues (RVDs). Fusion proteins of TAL effectors and nucleases (TALENs) can make targeted double-stranded breaks in cellular DNA that can be used to make specific genetic modifications to cells. In some embodiments, the agent is a TALEN polypeptide/protein or fragment or derivative thereof targets one or more TMIGD2 DNA sequences. In some embodiments, the Repeat Variable Diresidue (RVD) portion of the TALEN has been engineered to target one or more TMIGD2 DNA sequences. In some embodiments, the TALEN-based agent is a nucleic acid encoding one or more TALEN protein. In some embodiments, the nucleic acid is in a plasmid. In some embodiments, the nucleic acid is mRNA.

In some embodiments, the TALEN protein is expressed in a cell and induces a site-specific double stranded DNA break in one or more TMIGD2 gene. In some embodiments, the TALEN protein introduces a donor sequence, wherein the donor sequence partially or completely replaces the TMIGD2 gene, thereby silencing or inactivating the TMIGD2 gene. In some embodiments, the TALEN is a left TALEN and further comprising a right TALEN that cooperates with the left TALEN to make the double strand break in the TMIGD2 gene. In another embodiment, the nucleic acid encoding the TALEN and/or the nucleic acid donor sequence is part of a vector or plasmid. In some embodiment, the TALEN includes a spacer (e.g., the spacer sequence is 12 to 30 nucleotides in length).

Methods of engineering a TALEN to bind to specific nucleic acids are described in Cermak, et al, Nucl. Acids Res. 1-1 1 (2011). US Published Application No. 2011/0145940 discloses TAL effectors and methods of using them to modify DNA. Miller et al. Nature Biotechnol 29:143 (2011) describes the generation of TALENs for site-specific nuclease architecture by linking TAL truncation variants to the catalytic domain of Fok I nuclease. General design principles for TALEN binding domains can be found in, for example, WO 2011/072246. Each of these documents is incorporated herein in its entirety.

In some embodiments, the TALEN-based agent targets a nucleotide sequence of a TMIGD2. In some embodiments, the TALEN-based agent targets a nucleotide sequence that is conserved across more than one strain of TMIGD2. In some embodiments, the TALEN-based agent targets a TMIGD2 pol, env, and/or gag gene. In some embodiments, the TALEN-based agent targets a TMIGD2 pol gene. In some embodiments, the TALEN-based agent targets the sequence encoding the catalytic core of a TMIGD2 pol gene.

### II. ZFN-Based Agents

In some embodiments, the agent gene editing agent is a zinc finger nuclease (ZFN)-based agent. In some embodiments, the ZFN-based agent is one or more ZFN polypeptides or biologically active fragments or derivatives thereof, or one more nucleic acids encoding one or more ZFN polypeptides or fragments or derivatives thereof. ZFNs are artificial restriction enzymes generated by fusing a zinc finger DNA-binding domain to a nuclease. Zinc finger domains can be engineered to target specific desired DNA sequences and this enables zinc-finger nucleases to target unique sequences within complex genomes. The DNA-binding domains of individual ZFNs typically contain between three and six individual zinc finger repeats and can each recognize between 9 and 18 base pairs (bp). If the zinc finger domains perfectly recognize a 3 basepair DNA sequence to generate a 3-finger array, that array can recognize a 9 basepair target site. In some embodiments, either 1-finger or 2-finger modules are utilized to generate zinc-finger arrays with six or more individual zinc fingers. Because the specificities of individual zinc fingers can overlap and can depend on the context of the surrounding zinc fingers and DNA, ZFNs may not be useful for targeting specific TMIGD2.

Numerous selection methods have been developed to generate zinc-finger arrays capable of targeting desired sequences. In some embodiments, initial selection efforts utilize phage display to select proteins that bind a given DNA target from a large pool of partially randomized zinc-finger arrays. In some embodiments, yeast one-hybrid systems, bacterial one-hybrid and two-hybrid systems (e.g., the “OPEN” system), and mammalian cells may be used to select proteins that bind a given DNA. In particular, the OPEN system combines pre-selected pools of individual zinc fingers that were each selected to bind a given triplet and then utilizes a second round of selection to obtain 3-finger arrays capable of binding a desired 9-bp sequence.

In some embodiments, the process for editing the TMIGD2 gene sequence comprises introducing into a cell at least one nucleic acid encoding a zinc finger nuclease that recognizes the TMIGD2 sequence in the genome and is able to cleave a site in the TMIGD2 gene sequence. In some embodiments, process further comprises introducing at least one donor polynucleotide comprising a sequence for integration flanked by an upstream sequence and a downstream sequence that share substantial sequence identity with either side of the cleavage site. In some embodiments, process further comprises introducing at least one exchange polynucleotide comprising a sequence that is substantially identical to a portion of the genomic TMIGD2 sequence at the cleavage site and which further comprises at least one nucleotide change. In some embodiments, the cell is cultured to allow expression of the zinc finger nuclease such that the zinc finger nuclease introduces a double-stranded break into the genomic TMIGD2 sequence. In some embodiments, the double-stranded break is repaired by a non-homologous end-joining repair process such that a silencing or inactivating mutation is introduced into the chromosomal sequence. In some embodiments, the double-stranded break is repaired by a homology-directed repair process such that the sequence in the donor polynucleotide is integrated into the genomic TMIGD2 sequence or the sequence in the exchange polynucleotide is exchanged with the portion of the chromosomal sequence.

In some embodiments, the zinc finger nuclease targets a nucleotide sequence of a TMIGD2. In some embodiments, the zinc finger nuclease targets a nucleotide sequence that is conserved across more than one strain of TMIGD2. In some embodiments, the zinc finger nuclease targets a TMIGD2 pol, env, and/or gag gene. In some embodiments, the zinc finger nuclease targets a TMIGD2 pol gene. In some embodiments, the zinc finger nuclease targets the sequence encoding the catalytic core of a TMIGD2 pol gene.

### III. CRISPR-Based Agents

In some embodiments, the gene editing agent is a CRISPR-based agent. In some embodiments, the CRISPR-based agent comprises one or more polynucleotides involved in the expression of or directing the activity of CRISPR-associated genes, including but not limited to sequences encoding a nuclease gene (e.g., a gene encoding Cas9, Cas12a, or Cas13a), a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. In some embodiments, the CRISPR-based agent comprises a polynucleotide encoding at least one CRISPR protein and one or more guide RNAs (gRNAs). In some embodiments, the one or more gRNAs comprise a sequence cognate to a PERV polynucleotide sequence and capable of binding to a protospacer adjacent motif (“PAM”). In some embodiments, the PAM includes the sequence NGG or NNGRRT.

In some embodiments, the agent is a CRISPR-based polypeptide or fragment or derivative thereof that targets one or more TMIGD2 DNA sequences. In some embodiments, the CRISPR-based agent is characterized by elements that promote the formation of a CRISPR complex at the site of TMIGD2 DNA or RNA sequences. In some embodiments, the CRISPR-based agent is one or more CRISPR/Cas endonuclease or biologically active fragments or derivatives thereof, or one more nucleic acids encoding one or more CRISPR/Cas polypeptides or fragments or derivatives thereof. In some embodiments, the CRISPR/Cas endonuclease or a derivative thereof is from the CRISPR Type I system. In some embodiments, the CRISPR/Cas endonuclease or a derivative thereof is from the CRISPR Type II system. In some embodiments, the CRISPR/Cas endonuclease or a derivative thereof is from the CRISPR Type III system. In some embodiments, the CRISPR/Cas endonuclease or a derivative thereof is from the CRISPR Type IV system. In some embodiments, the CRISPR/Cas endonuclease or a derivative thereof is from the CRISPR Type V system. In some embodiments, the CRISPR/Cas endonuclease or a derivative thereof is from the CRISPR Type VI system. In some embodiments, the CRISPR/Cas endonuclease or a derivative thereof is from the CRISPR Type IIA, Type IIB or Type IIC systems. In some embodiments, the CRISPR/Cas endonuclease or a derivative thereof is from the CRISPR Type IIC system. In some embodiments, the type II CRISPR/Cas endonuclease is Cas9, or a derivative thereof. In some embodiments, the CRISPR/Cas endonuclease or a derivative thereof is a type V CRISPR/Cas endonuclease, such as Cpf1 (Cas12a), or a derivative thereof. In some embodiments, a CRISPR/Cas endonuclease or a derivative thereof is a type VI CRISPR/Cas endonuclease, such as Cas13a, or a derivative thereof. In some embodiments, the site-directed modifying polypeptide is a Type III-B Cmr complex, e.g., a Type III-B Cmr complex derived from Pyrococcus furiosus, Sulfolobus solfataricus, or Thermus thermophilus. See, e.g., Hale, C. R. et al. Genes & Development, 2014, 28:2432-2443, and Makarova K. S. et al. Nature Reviews Microbiology, 2015, 13, 1-15.

In particular embodiments, the CRISPR-based agent utilizes the Type II Cas9 endonuclease. In some embodiments, the CRISPR-based agent comprises the Type II Cas9 endonuclease and an additional polynucleotide. In some embodiments, the additional polynucleotide is a tracrRNA, crRNA (also referred to as a “tracr-mate RNA) and/or a synthetic single guide RNA (sgRNA). See, e.g., Jinek, M., et al. (2012) Science, 337, 816-821.

In some embodiments, the CRISPR-based agent is a Cas protein that lacks the ability to cleave double stranded DNA. In some embodiments, the Cas protein is capable of only cleaving a single strand of DNA, i.e., the Cas protein is a “nickase.” In some embodiments, the Cas protein is incapable of cleaving either strand of DNA. In some embodiments, the Cas protein is a Cas9 protein that has been mutated such that it is a nickase or such that it lacks the ability to cleave either strand of DNA. In some embodiments, the Cas9 protein has a D10A and/or an H840A mutation. In some embodiments, the agent is a polynucleotide that encodes for a Cas9 protein having a D10A and/or an H840A mutation. See, e.g., Cong L., et al. (2013) Science, 339, 819-823; Jinek, M., et al. (2012) Science, 337, 816-821; Gasiunas, G., et al. (2012) Proc. Natl. Acad. Sci. USA, 109, E2579-2586; and Mali, P., et al. (2013) Science, 339, 823-826; each of which is incorporated by reference herein in its entirety.

In some embodiments, the CRISPR-based agent comprises a gRNA. In some embodiments, the gRNA targets a nucleotide sequence of TMIGD2. In some embodiments, the gRNA targets a TMIGD2pol, env, and/or gag gene. In some embodiments, the gRNA targets a TMIGD2 pol gene. In some embodiments, the gRNA targets the sequence encoding the catalytic core of a TMIGD2 pol gene. In some embodiments, the gRNA targets a non-catalytic core region of a TMIGD2 pol gene. In some embodiments, the non-catalytic core region of a TMIGD2 pol gene is upstream of the catalytic core region of a TMIGD2 pol gene.

In some embodiments, the agent comprises at least two guide RNAs, at least three guide RNAs, at least four guide RNAs, at least five guide RNAs, at least six guide RNAs, at least seven guide RNAs, at least eight guide RNAs, at least nine guide RNAs, at least 10 guide RNAs, at least 11 guide RNAs, at least 12 guide RNAs, at least 13 guide RNAs, at least 14 guide RNAs, at least 15 guide RNAs, at least 60 guide RNAs, at least 17 guide RNAs, at least 18 guide RNAs, at least 19 guide RNAs, at least about 20 guide RNAs, at least 3 about 0 guide RNAs, at least about 40 guide RNAs, at least about 50 guide RNAs, at least about 60 guide RNAs, at least about 70 guide RNAs, at least about 80 guide RNAs, at least about 90 guide RNAs, at least about 100 guide RNAs, or more.

In some embodiments, the CRISPR-based agent is a combination of any of the CRISPR-based polypeptides/proteins and CRISPR-based polynucleotides disclosed herein. For example, in some embodiments, the CRISPR-based agent comprises a Cas endonuclease and guide RNA. In some embodiments, the CRISPR-based agent comprises a Cas endonuclease, tracrRNA and tracr-mate sequence. In some embodiments, the tracrRNA and tracr-mate sequence are engineered such that they are in the same molecule. In some embodiments, the CRISPR-based agent is one or more polynucleotides encoding any of the foregoing.

In some embodiments, the CRISPR-based agent is a chimeric RNA such as a CRISPR-Cas system RNA. In some embodiments, the CRISPR-based agent has at least one second guide sequence capable of hybridizing to an RNA sequence of the CRISPR-Cas system or a nucleic acid molecule for expression of a component of the CRISPR-Cas complex to diminish or eliminate functional expression of the system or complex, whereby the system or complex can be self-inactivating; and, the second guide sequence can be capable of hybridizing to a nucleic acid molecule for expression of the CRISPR enzyme.

In some embodiments, the disclosure provides methods for using any of the CRISPR-based agents disclosed herein. In some embodiments, the disclosure provides an effective means for modifying a TMIGD2 polynucleotide sequences by utilizing any of the CRISPR-based agents disclosed herein. The CRISPR complex of the invention has a wide variety of utilities including modifying (e.g., deleting, inactivating) TMIGD2 polynucleotide sequences in different types of cells from various tissues and organs. As such the CRISPR complex of the invention has a broad spectrum of applications in, e.g., gene or genome editing.

In some embodiments, the disclosure provides an in vivo method of genomic editing comprising providing a quantity of one or more vectors each encoding at least one CRISPR protein and one or more guide RNAs (gRNAs), and administering the one or more vectors to a mammal, wherein in vivo expression of the one or more vectors includes binding of the CRISPR protein to a TMIGD2 locus cognate to the gRNA and in vivo generation of a double stranded break (DSB) in a population of cells in the mammal, wherein in vivo homologous recombination (HR) of the DSB results in editing of the genome of a population of cells in the mammal. In some embodiments, the CRISPR protein is Cas9 and the one or more gRNAs comprise a sequence capable of binding to a protospacer adjacent motif (“PAM”). In some embodiments, HR includes non-homologous end joining (NHEJ) introducing missense or nonsense of a protein expressed at the PERV locus.

In some embodiments, the CRISPR-based agent further comprises a portion that modulates TMIGD2 expression. In some embodiments, the CRISPR-based agent is a fusion protein that comprises a transcription repressor domain.

### E. Combination Therapies

In some embodiments, an agent that inhibits TMIGD2 expression, activity, or both is administered with at least a second or additional agent. In certain of these embodiments, an agent that inhibits TMIGD2 expression, activity, or both is administered in combination with an immune checkpoint inhibitor. Immune checkpoint proteins are well-known in the art and include, without limitation, CTLA-4, PD-I, VISTA, B7-H2, B7-H3, PD-L1, B7-H4, B7-H6, 2B4, ICOS, HVEM, PD-L2, CD160, gp49B, PIR-B, KIR family receptors, TIM-I, TIM-3, TIM-4, LAG-3, BTLA, SIRPalpha (CD47), CD48, 2B4 (CD244), B7.1, B7.2, ILT-2, ILT-4, TIGIT, HHLA2, KIR3DL3, and A2aR (see, for example, WO 2012/177624, which is hereby incorporated by reference in its entirety). Inhibition of one or more immune checkpoint inhibitors can block or otherwise neutralize inhibitory signaling to thereby upregulate an immune response in order to more efficaciously treat cancer.

In certain embodiments, an agent that inhibits TMIGD2 expression, activity, or both is administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g, standard-of-care treatments for cancer well-known to the skilled artisan), either consecutively with, pre- or post-conventional therapy. For example, agents described herein can be administered with a therapeutically effective dose of chemotherapeutic agent. Such a chemotherapeutic agent may include, but is not limited to: platinum compounds, cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents e.g., FLAG or CHOP, may also be used. FLAG comprises fludarabine, cytosine arabinoside (Ara-C) and G-CSF. CHOP comprises cyclophosphamide, vincristine, doxorubicin, and prednisone. The foregoing examples of chemotherapeutic agents are illustrative, and are not intended to be limiting.

In another embodiment, an agent that inhibits TMIGD2 expression, activity, or both is administered in combination with radiation therapy. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfm (BPD-MA), phthalocyanine, photosensitizer Pc4, demethoxy-hypocrellin A; and 2B A-2-DMHA.

In certain embodiments, an agent that inhibits TMIGD2 expression, activity, or both is administered in combination with hyperthermia, photodynamic therapy, and/or surgery. In such embodiments, treatment with hyperthermia comprises or is local hyperthermia (e.g., external, intraluminal, or interstitial hyperthermia), regional hyperthermia (e.g., deep tissue hyperthermia, regional perfusion, or (continuous hyperthermic peritoneal perfusion), or whole-body hyperthermia. In some embodiments, a photodynamic therapy comprises or is administration of photosensitizers, such as hematoporphyrin and its derivatives, Verteporfin (BPD-MA), phthalocyanine, photosensitizer Pc4, demethoxy-hypocrellin A, 2BA-2-DMHA, or a combination thereof. In some embodiments, surgery comprises or is surgery to remove cancerous or precancerous tissue. In some embodiments, a transplant comprises or is a stem cell transplant or an organ transplant.

In another embodiment, an agent that inhibits TMIGD2 expression, activity, or both is administered in combination with hormone therapy. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g, all-trans retinoic acid (ATRA)); vitamin D3 analogs; antigestagens (e.g, mifepristone, onapristone), or antiandrogens (e.g, cyproterone acetate).

In certain embodiments, an agent that inhibits TMIGD2 expression, activity, or both is administered in combination with immunomodulatory interleukins, such as IL-2, IL-6, IL-7, IL-12, IL-17, IL-23, and the like, as well as modulators thereof (e.g., blocking antibodies or more potent or longer lasting forms). In another embodiment, the agent that inhibits TMIGD2 expression, activity, or both is administered in combination with immunomodulatory cytokines, such as interferons, G-CSF, imiquimod, TNF alpha, and the like, as well as modulators thereof (e.g., blocking antibodies or more potent or longer lasting forms). In another embodiment, the agent that inhibits TMIGD2 expression, activity, or both is administered in combination with immunomodulatory chemokines, such as CCL3, CCL26, and CXCL7, and the like, as well as modulators thereof (e.g., blocking antibodies or more potent or longer lasting forms). In another embodiment, the agent that inhibits TMIGD2 expression, activity, or both is administered in combination with immunomodulatory molecules targeting immunosuppression, such as STAT3 signaling modulators, NFkappaB signaling modulators.

In certain embodiments, an agent that inhibits TMIGD2 expression, activity, or both is administered in combination with immunomodulatory drugs, such as immunocytostatic drugs, glucocorticoids, cytostatics, immunophilins and modulators thereof (e.g, rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fmgolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic tri oxide, dehydroxymethylepoxyquinomycin (DHMEQ), 13C (indole-3-carbinol)/DIM (di-indolmethane) (13C/DIM), Bay 11-7082, luteolin, cell permeable peptide SN-50, IKBa-super repressor overexpression, NFKB decoy oligodeoxynucleotide (ODN), or a derivative or analog of any thereof.

In certain embodiments, an agent that inhibits TMIGD2 expression, activity, or both is administered in combination with immunomodulatory antibodies or proteins, such as antibodies that bind to CD40, Toll-like receptor (TLR), 0X40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CDI47 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-I antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-I receptor antagonist, anakinra, an anti-IL-5 antibody, mepolizumab, an IgE inhibitor, omalizumab, talizumab, an IL12 inhibitor, an IL23 inhibitor, ustekinumab, and the like.

In certain embodiments, an agent that inhibits TMIGD2 expression, activity, or both is administered in combination with adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, natural killer (NK) cells, autologous immune enhancement therapy (AIET), cancer vaccines, antigen presenting cells, and/or a combination thereof. Such cell-based immunotherapies can be further modified to express one or more gene products to further modulate immune responses, such as expressing cytokines like GM-CSF, and/or to express tumor-associated antigen (TAA) antigens, such as Mage-I, gp-IOO, patient-specific neoantigen vaccines, and the like.

### Methods of Making

The present disclosure, among other things, provides methods of making antibodies or antigen-binding fragments thereof described herein. In some embodiments, an antibody or antigen-binding fragment thereof described herein is identified using a display technology, such as yeast display, phage display, or ribosome display. In some embodiments, an antibody or antigen-binding fragment thereof described herein is identified using a hybridoma library (e.g., a mammalian hybridoma library, e.g., a mouse hybridoma library), followed by supernatant screening.

Combinatorial methods for generating antibodies or antigen-binding fragments thereof are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibody Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, each of which is hereby incorporated by reference in its entirety).

An antibody or antigen-binding fragment thereof described herein can be derived from other species. A humanized antibody is an antibody produced by recombinant DNA technology, in which some or all amino acids of a human immunoglobulin light chain or heavy chain that are not required for antigen binding (e.g., constant regions and/or framework regions of variable domains) are used to substitute for the corresponding amino acids from light chain or heavy chain of the cognate, nonhuman antibody. By way of example, a humanized version of a murine antibody to a given antigen has on both heavy and light chains: (1) constant regions of a human antibody; (2) FRs from the variable domains of a human antibody; and (3) CDRs from the murine antibody. Human FRs may be selected based on their highest sequence homology to mouse FR sequence. When necessary, one or more residues in human FRs can be changed to residues at corresponding positions in a murine antibody so as to preserve binding affinity of the humanized antibody to a target. This change is sometimes called “back mutation.” Similarly, forward mutations may be made to revert back to murine sequence for a desired reason, e.g. stability or affinity to a target. Humanized antibodies generally are generally less likely to elicit an immune response in humans as compared to chimeric human antibodies because the former contain considerably fewer non-human components.

Methods for humanizing non-human antibodies are well known in the art. Suitable methods for making humanized antibodies in accordance with the present disclosure are described in, e.g., Winter EP 0 239 400; Jones et al., Nature 321:522-525 (1986); Riechmann et al., Nature 332:323-327 (1988); Verhoeyen et al., Science 239:1534-1536 (1988); Queen et al., Proc. Nat. Acad. ScL USA 86:10029 (1989); U.S. Pat. No. 6,180,370; and Orlandi et al., Proc. Natl. Acad. Sd. USA 86:3833 (1989); the disclosures of each of which are incorporated herein by reference in their entireties. Generally, transplantation of non-human (e.g., murine) CDRs onto a human antibody is achieved as follows. cDNAs encoding VH and VL are isolated from a hybridoma, and nucleic acid sequences encoding VH and VL including CDRs are determined by sequencing. Nucleic acid sequences encoding CDRs are inserted into corresponding regions of a human antibody VH or VL coding sequences and attached to human constant region gene segments of a desired isotype (e.g., yl for CH and K for CL). Humanized heavy and light chain genes are co-expressed in mammalian host cells (e.g., CHO or NSO cells) to produce soluble humanized antibody. To facilitate large-scale production of antibodies, it is often desirable to select for a high expressor using, for example, a DHFR gene or GS gene in the producer line.

An antibody or antigen-binding fragment thereof described herein can be or comprise a human antibody or antigen-binding fragment thereof. Completely human antibodies may be particularly desirable for therapeutic treatment of human subjects. Human antibodies can be made by a variety of methods known in the art including phage display methods described above using antibody libraries derived from human immunoglobulin sequences (see, e.g., U.S. Pat. Nos. 4,444,887 and 4,716,111; and PCT publications WO 98/46645, WO 98/60433, WO 98/24893, WO 98/16664, WO 96/34096, WO 96/33735, and WO 91/10741; each of which is incorporated herein by reference in its entirety). Techniques are also available for the preparation of human monoclonal antibodies in, e.g., Cole et al., Monoclonal Antibodies and Cancer Therapy, Alan R. Riss, (1985); and Boerner et al., J. Immunol., 147 (1): 86-95, (1991), each of which is incorporated herein by reference in its entirety.

An antibody or antigen-binding fragment thereof described herein can be or comprise a chimeric antibody or antigen-binding fragment thereof. Illustrative methods of making chimeric antibodies are described, for example, in U.S. Pat. No. 4,816,567; and Morrison et al., Proc. Natl. Acad. Sci. USA, 1984, 81:6851-6855; each of which is incorporated by reference in its entirety. In some embodiments, a chimeric antibody is made by using recombinant techniques to combine a non-human variable region (e.g., a variable region derived from a mouse, rat, hamster, rabbit, or non-human primate, such as a monkey) with a human constant region.

Any suitable method can be used to introduce variability into one or more polynucleotide sequences encoding an antibody or antigen-binding fragment thereof described herein, including error-prone PCR, chain shuffling, and oligonucleotide-directed mutagenesis such as trinucleotide-directed mutagenesis (TRIM). In some embodiments, several CDR residues (e.g., 4-6 residues at a time) are randomized. CDR residues involved in antigen binding may be specifically identified, for example, using alanine scanning mutagenesis or modeling. CDR-H3 and CDR-L3 in particular are often targeted for mutation. Introduction of diversity into variable regions and/or CDRs can be used to produce a secondary library. A secondary library is then screened to identify antibody variants with improved affinity. Affinity maturation by constructing and reselecting from secondary libraries has been described, for example, in Hoogenboom et al., Methods in Molecular Biology, 2001, 178:1-37, incorporated by reference in its entirety.

### Compositions

Provided herein in certain embodiments are compositions comprising one or more of the agents used in the methods provided herein, as well as the use of these agents to inhibit TMIGD2 expression and/or activity.

In some embodiments, the compositions provided herein are pharmaceutical compositions comprising one or more agents that inhibit TMIGD2 expression and/or activity and a pharmaceutically acceptable excipient. Non-limiting examples of pharmaceutically acceptable excipients include, for example, those described in “Remington: The Science and Practice of Pharmacy”, 19th Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc. In some embodiments, the composition is suitable for administration to a subject, for example, a sterile composition. In some embodiments, the composition is suitable for administration to a human subject, for example, the composition is sterile and is free of detectable pyrogens and/or other toxins.

In some embodiments, the composition comprises other components, such as pharmaceutical grades of mannitol, lactose, starch, magnesium stearate, sodium saccharin, talcum, cellulose, glucose, sucrose, magnesium, carbonate, and the like. In some embodiments, the compositions comprise a pharmaceutically acceptable auxiliary substance as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

In some embodiments, the compositions are in an aqueous solution, powder form, granules, tablets, pills, suppositories, capsules, suspensions, sprays, and the like. The composition may comprise a pharmaceutically acceptable excipient, a pharmaceutically acceptable salt, diluents, carriers, vehicles and such other inactive agents well known to the skilled artisan. Vehicles and excipients commonly employed in pharmaceutical preparations include, for example, talc, gum Arabic, lactose, starch, magnesium stearate, cocoa butter, aqueous or non-aqueous solvents, oils, paraffin derivatives, glycols, etc. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions may be prepared using conventional techniques that may include sterile isotonic saline, water, 1,3-butanediol, ethanol, 1,2-propylene glycol, polyglycols mixed with water, Ringer's solution, etc. In one aspect, a coloring agent is added to facilitate in locating and properly placing the composition to the intended treatment site.

Compositions may include a preservative and/or a stabilizer. Non-limiting examples of preservatives include methyl-, ethyl-, propyl-parabens, sodium benzoate, benzoic acid, sorbic acid, potassium sorbate, propionic acid, benzalkonium chloride, benzyl alcohol, thimerosal, phenylmercurate salts, chlorhexidine, phenol, 3-cresol, quaternary ammonium compounds (QACs), chlorbutanol, 2-ethoxyethanol, and imidurea.

To control tonicity, the composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8, e.g., between 6.5 and 7.5, or between 7.0 and 7.8.

The composition can be administered by any appropriate route, which will be apparent to the skilled person depending on the disease or condition to be treated. Typical routes of administration include intravenous, intra-arterial, intramuscular, subcutaneous, intracranial, intranasal or intraperitoneal.

In some embodiments, the composition may include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

The composition can comprise a pharmaceutically acceptable excipient, a pharmaceutically acceptable salt, diluents, carriers, vehicles and such other inactive agents well known to the skilled artisan. Vehicles and excipients commonly employed in pharmaceutical preparations include, for example, talc, gum Arabic, lactose, starch, magnesium stearate, cocoa butter, aqueous or non-aqueous solvents, oils, paraffin derivatives, glycols, etc. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions may be prepared using conventional techniques that may include sterile isotonic saline, water, 1,3-butanediol, ethanol, 1,2-propylene glycol, polyglycols mixed with water, Ringer's solution, etc. In one aspect, a coloring agent is added to facilitate in locating and properly placing the composition to the intended treatment site.

As can be appreciated from the disclosure above, the present invention has a wide variety of applications. The invention is further illustrated by the following examples, which are only illustrative and are not intended to limit the definition and scope of the invention in any way.

### Example: Targeting TMIGD2 to Treat Hematologic Malignancies

The following example demonstrates that TMIGD2 is expressed in various human hematologic malignancies, is functionally important for leukemia-initiating cells, and is associated with worse overall survival of AML patients. This example also demonstrates that the knock down of TMIGD2 impairs AML stem cells maintenance and increases cell death of human hematologic malignancies. In addition, the example demonstrates that the treatment of anti-TMIGD2 monoclonal antibodies inhibit AML progress in vivo. Taken together, the results from this example suggest that targeting TMIGD2 expression can be used a method for treating hematologic malignancies.

### TMIGD2 is Highly Expressed in Various Hematologic Malignancies

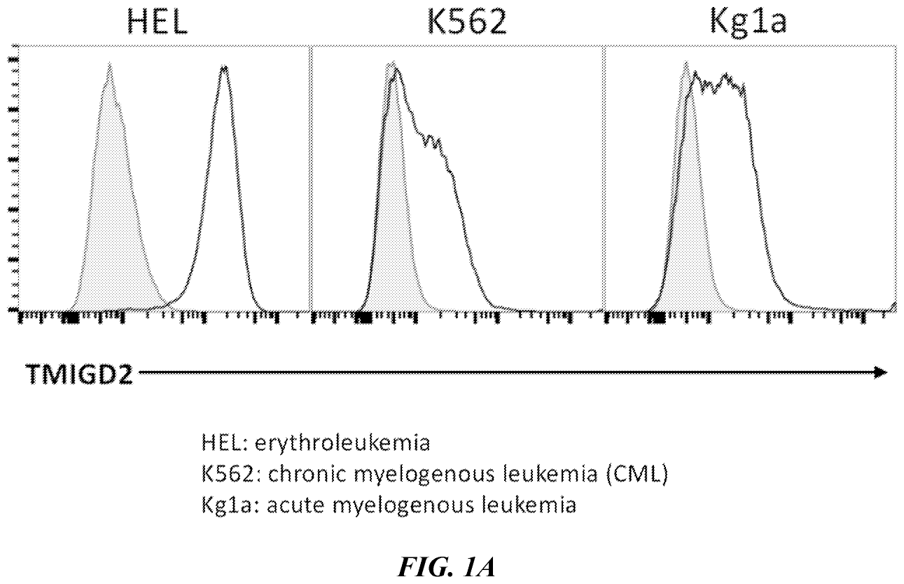

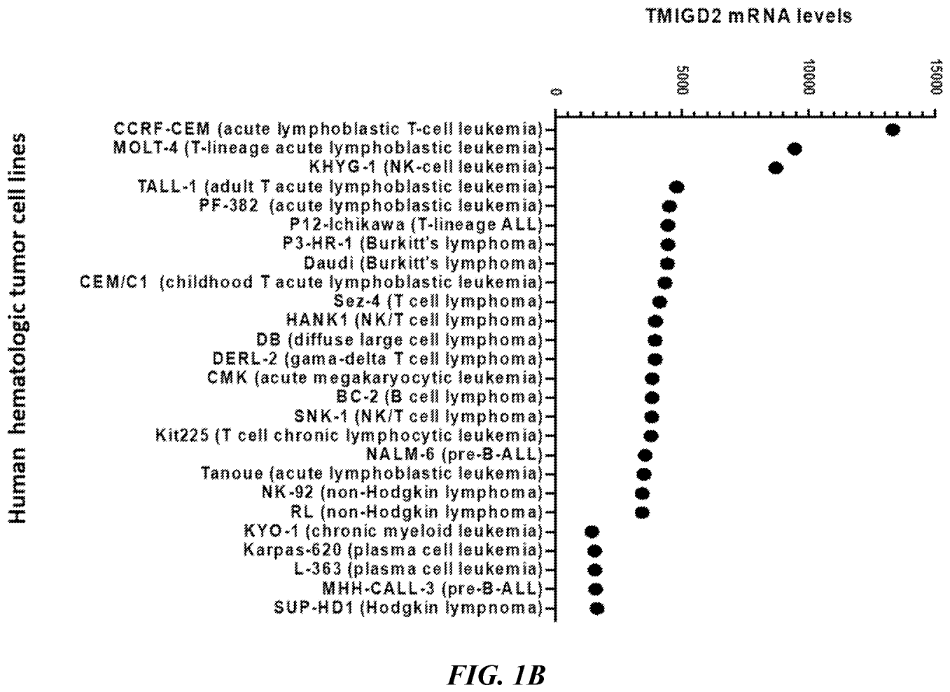

While TMIGD2 had been identified as a member of the CD28 family and a receptor for HHLA2, expression of TMIGD2 at the protein level in human tumor cells remained unknown. To examine the protein expression, fluorescence-activated cell sorting (FACS) and monoclonal (mAb) were used against TMIGD2 to examine TMIGD2 protein on various hematologic malignancies. The results showed that three tumor lines of human erythroleukemia (HEL), chronic myelogenous leukemia (K562), and acute myelogenous leukemia (Kg1a) expressed high levels of TMIGD2 protein on their cell surface (FIG. 1A). TMIGD2 mRNA was highly expressed in cell lines of human leukemia, lymphoma, multiple myeloma, etc. (FIG. 1B)

TMIGD2 mRNA, but not PD-L1/PD-1, is Highly Expressed in Human Acute Myeloid Leukemia (AML) and is Associated with Worse Overall Survival of Patients

The cancer genome atlas (TCGA) and genotype-tissue expression (GTEx) datasets contain 173 human acute myeloid leukemia (AML) samples and 70 normal bone marrow samples, the following analysis included studying the mRNA expression of the HHLA2/TMIGD2/KIR3DL3 pathways as well as the PDL1/PD-1 pathway.

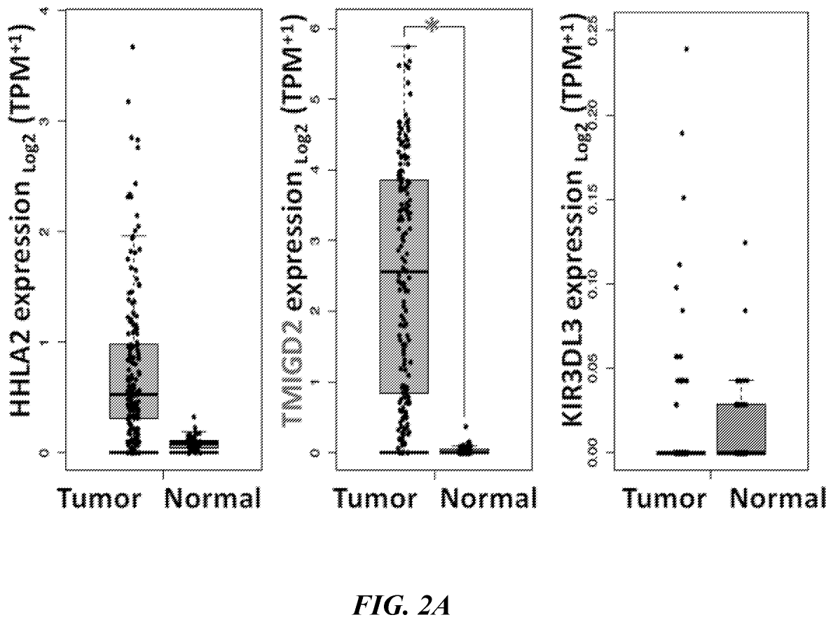

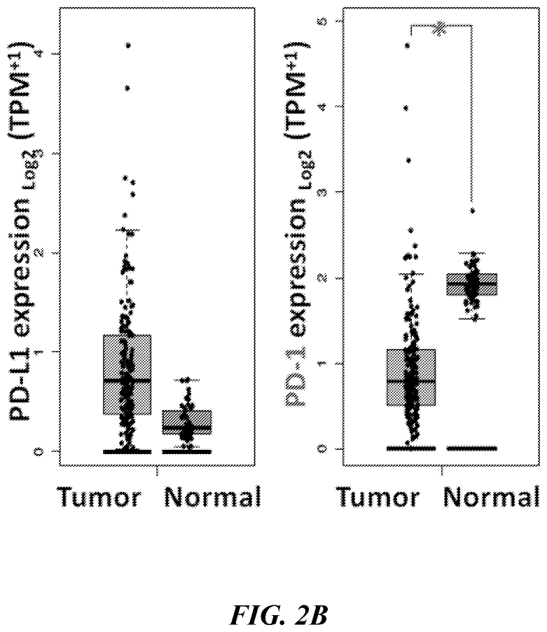

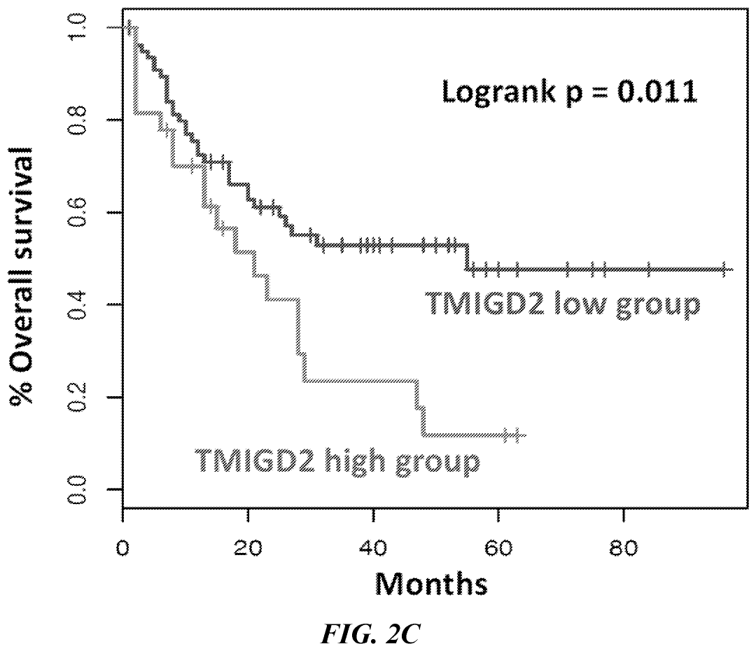

The analysis revealed that the level of TMIGD2 mRNA in AML samples was significantly higher than that of normal bone marrow samples (FIG. 2A), HHLA2 expression was also higher in AML samples but did not reach statistical significance (FIG. 2A), whereas KIR3DL3 expression is very low. In contrast to TMIGD2, the level of PD-1 mRNA in AML samples was significantly lower than that of normal bone marrow samples (FIG. 2B).

The AML samples were further separated into two groups according to their TMIGD2 expression levels: the TMIGD2 high group (top 25%) and the TMIGD2 low group (the rest 75%). The result revealed that the TMIGD2 high group had significantly poorer overall survival than the TMIGD2 low group (p=0.011) (FIG. 2C). Taken together, these results suggest that TMIGD2, but not the long-standing pathway of PD-L1/PD-1, is highly expressed in human AML and is associated with worse overall survival of patients.

### TMIGD2 is Highly Expressed on AML Stem/Progenitor Cells

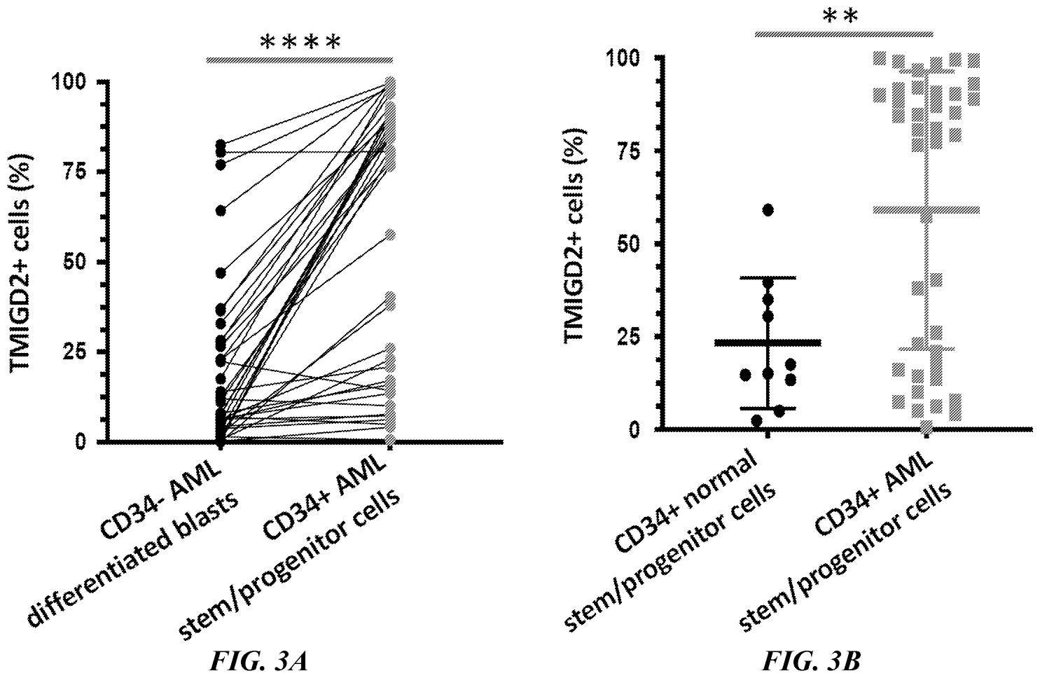

FACS was used to determine TMIGD2 protein expression on peripheral blood cells form 40 AML patients, cord blood mononuclear cells from 5 heathy donors, and bone marrow cells from 5 heathy adults. The study revealed that TMIGD2 positive cells in CD34+AML stem/progenitor cells were significantly higher than CD34-AML differentiated blasts (FIG. 3A, P<0.0001). Furthermore, TMIGD2 positive cells in CD34+AML stem/progenitor cells were significantly higher than CD34+ normal stem/progenitor cells in cord blood/bone marrow mononuclear cells from healthy donors (FIG. 3B, P<0.01).

### TMIGD2 Enriches for Functional Leukemia-Initiating Cells

As TMIGD2 is overexpressed on AML stem cells (FIGS. 3A-3B), two sets of experiments to directly compare the frequency of leukemia-initiating cells between TMIGD2+ and TMIGD2-AML stem cells (CD45dimSSClowLin-(CD3-CD14-CD19-) CD34+CD38−) were performed.

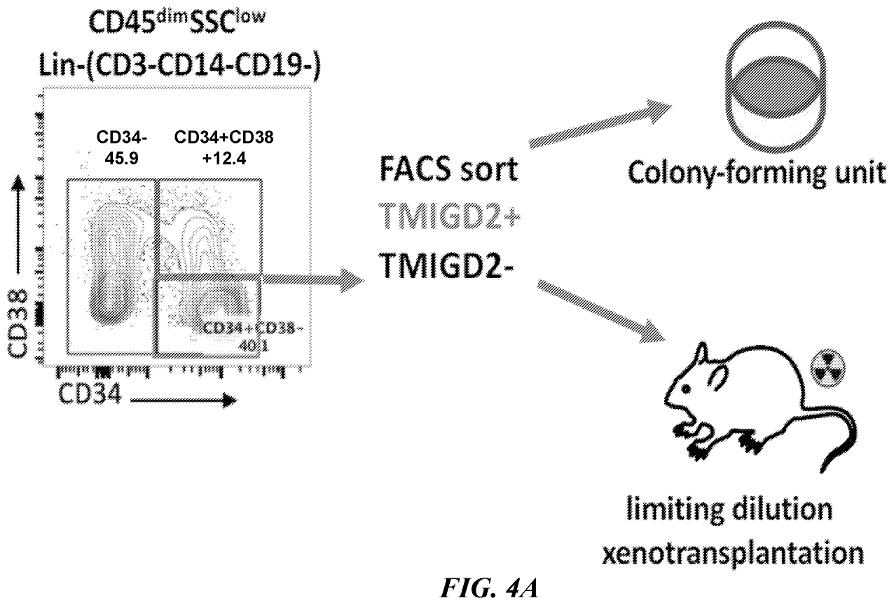

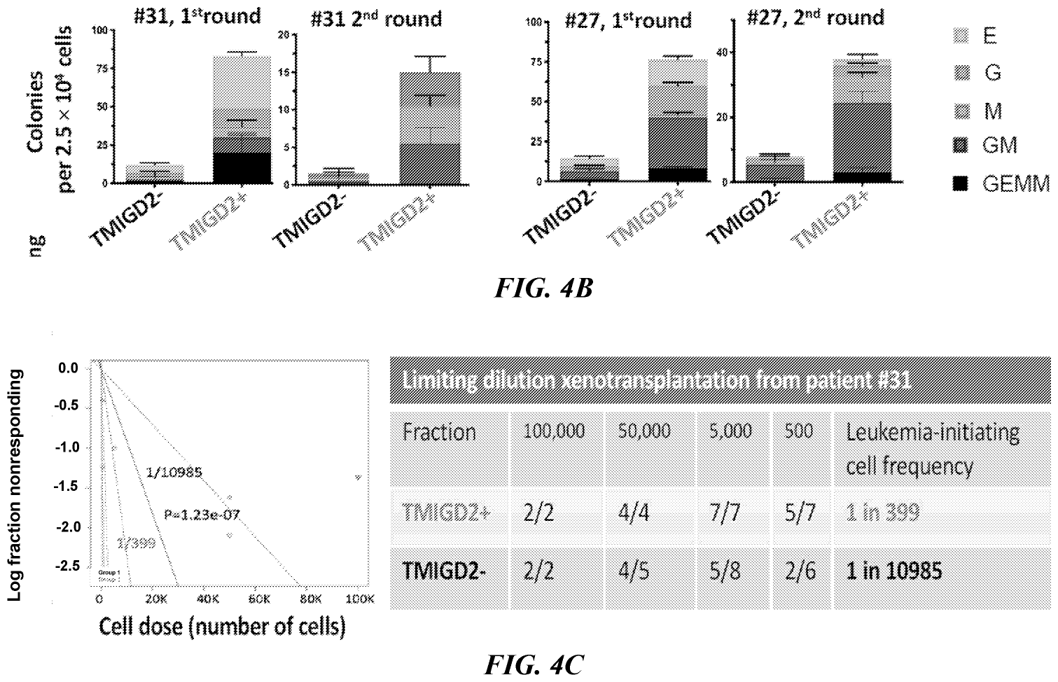

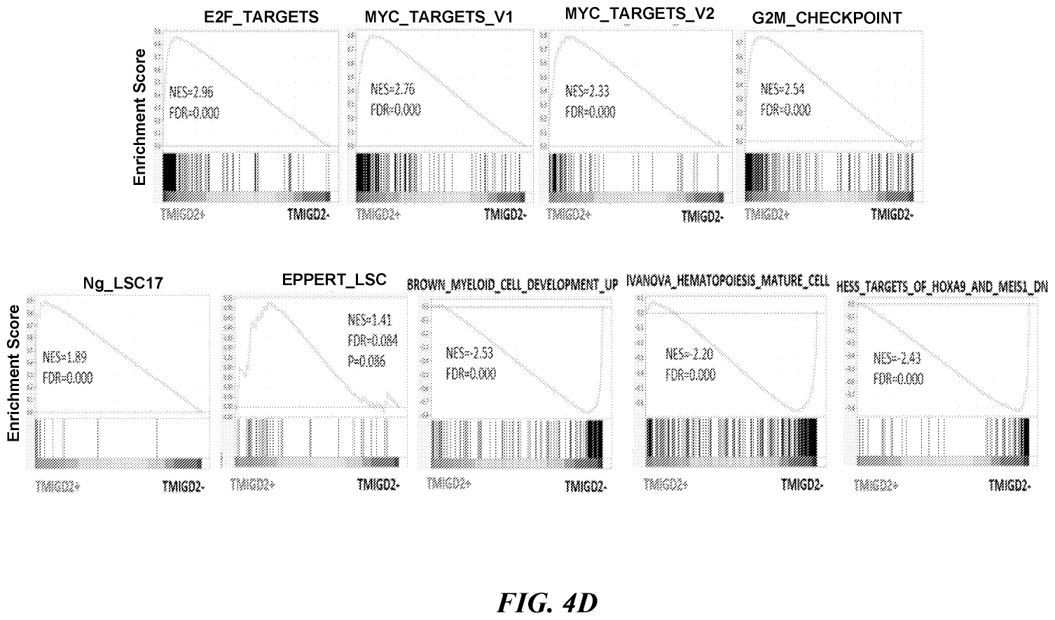

First, TMIGD2+ and TMIGD2-AML stem cells from AML samples were sorted by FACS (FIG. 4A), and then an in vitro colony-forming unit (CFU) assay was performed by plating the purified cells in methylcellulose-based media. The colonies formed were enumerated and characterized according to their unique morphology. The second re-plating was carried out by collecting colony cells to evaluate their self-renewal capacity. Compared to TMIGD2-AML stem cells from the same patient, TMIGD2+ AML stem cells formed much higher CFU numbers of colonies after 14 days culture in both first round and second round cultures (FIG. 4B).

Second, TMIGD2+ and TMIGD2-AML stem cells from the same AML sample were sorted by FACS, and then in vivo limiting dilution xenotransplantation experiments were carried out by transplanting sublethally irradiated NSG mice with the sorted two subpopulations. After >12 weeks bone marrow cells in these NSG mice were analyzed by FACS to determine the lymphoid and myeloid engraftment.

It was found that the frequency of leukemia-initiating cells between TMIGD2+ and TMIGD2-AML stem cells from the same patient (patient #31) are 1/399 and 1/10985, respectively (FIG. 4C). These data demonstrate that TMIGD2 enriches for functional leukemia-initiating cells.

RNA-seq comparison between CD34+TMIGD2+ subpopulation and CD34+TMIGD2− subpopulation demonstrated TMIGD2+ AML stem cells were associated with the established leukemic stem cells (LSC) and 17-gene stemness signatures (FIG. 4D).

### Knock-Down of TMIGD2 Impairs AML Stem Cells Maintenance

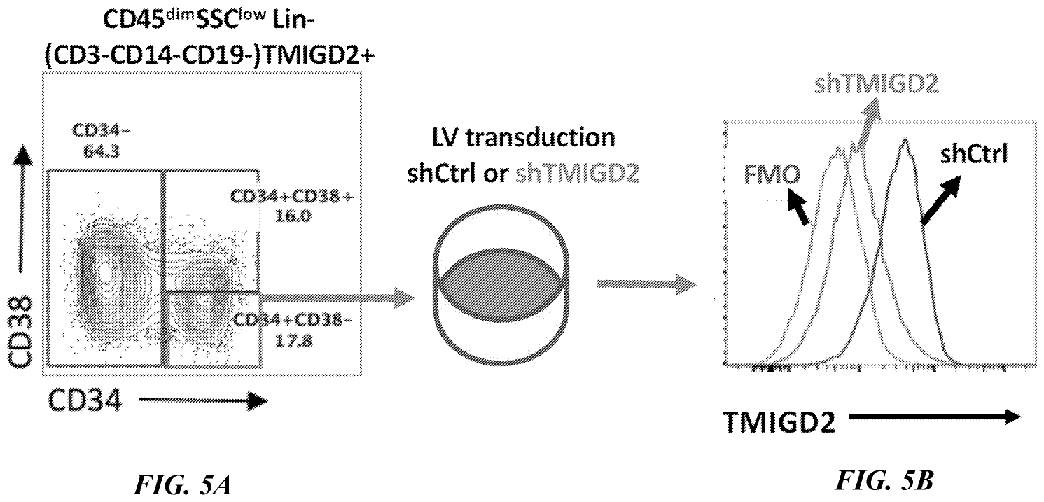

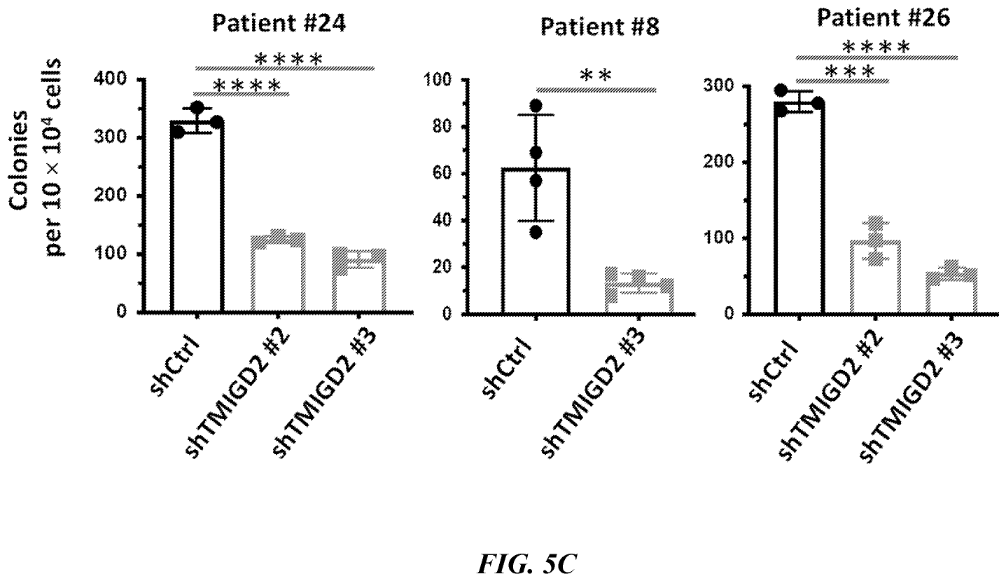

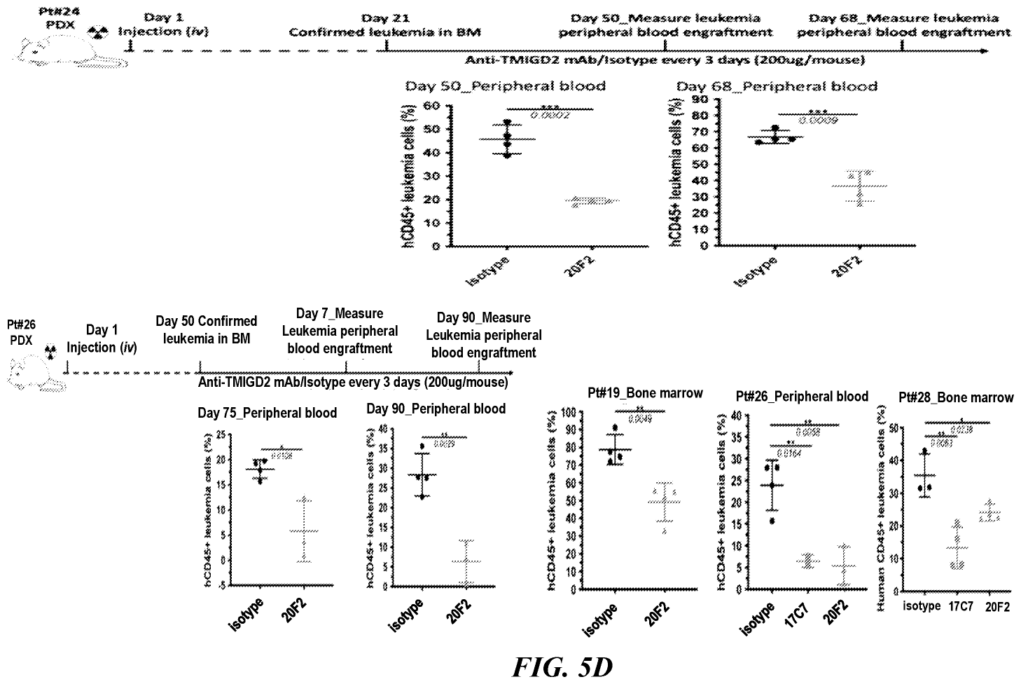

TMIGD2 is overexpressed on AML stem cells (FIGS. 3A-3B) and is associated with worse overall survival of patients (FIGS. 2A-2C), suggesting TMIGD2 plays a critical role in AML stem cells. To dissect the function of TMIGD2, TMIGD2+ AML stem cells (CD45dimSSClowLin-(CD3−CD14−CD19−) CD34+CD38−) were sorted from AML peripheral blood using FACS, transduced with lentivirus expressing Scramble control shRNA (shCtrl) or TMIGD2 specific shRNA (shTMIGD2) (FIG. 5A) and sorted for GFP at day 3 post-transduction. As shown in FIG. 5B, compared to shCtrl, shTMIGD2 reduced the majority of TMIGD2 expression on AML stem cells. A CFU assay was then performed and it was found that TMIGD2 knock-down on AML stem cells significantly reduced colony formation in all three AML patient samples (FIG. 5C). These results demonstrate that TMIGD2 is functionally important for AML stem cells maintenance and that targeting TMIGD2 reduces the survival of AML stem cells.

### Knock-Down of TMIGD2 Increase Cell Death of Human Hematologic Malignancies

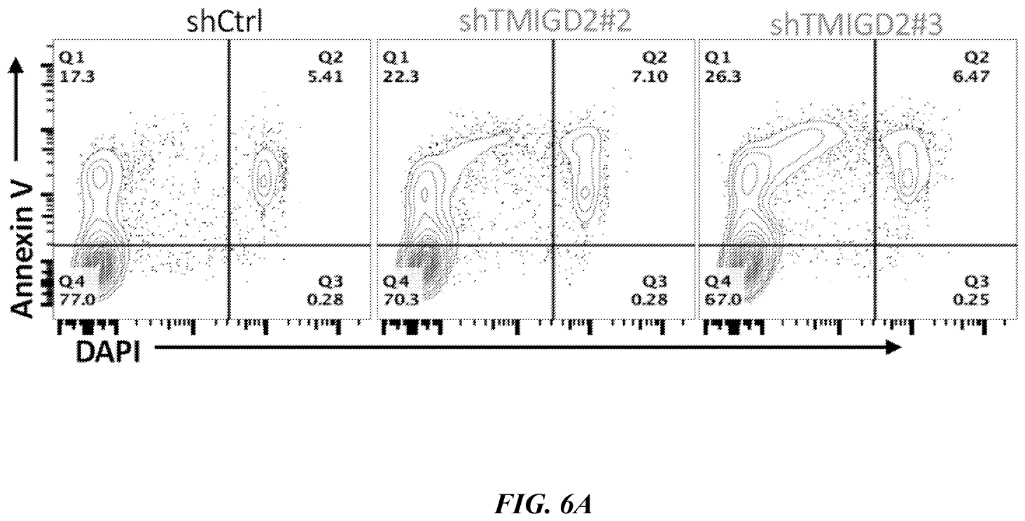

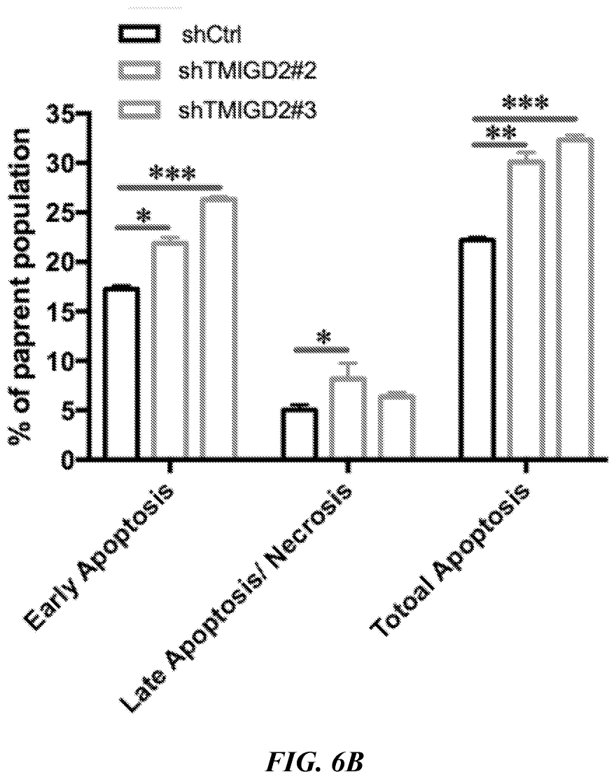

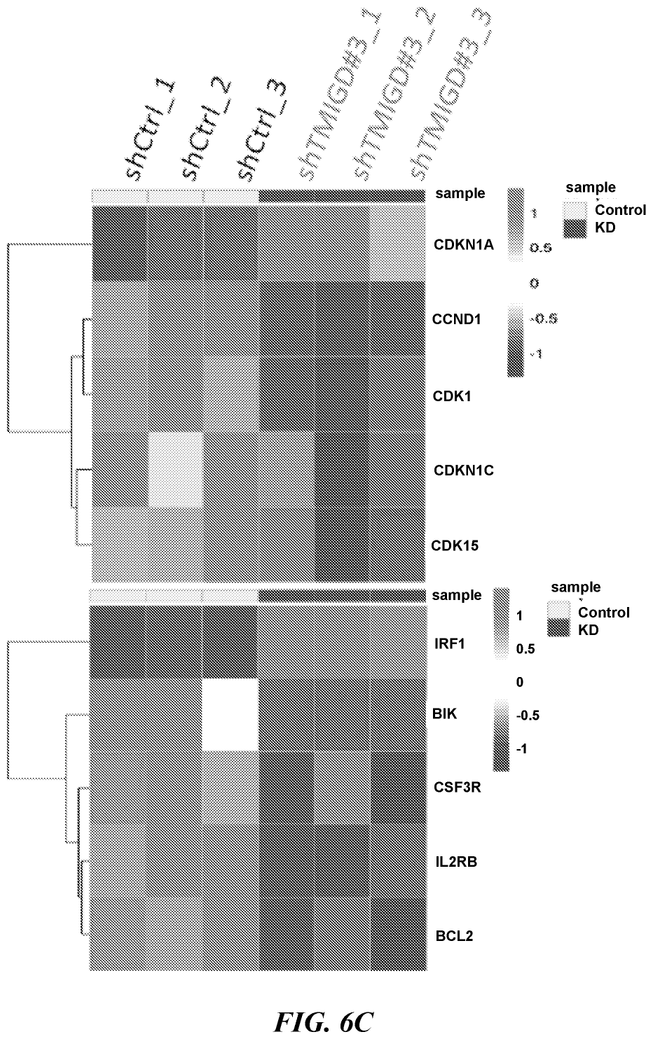

To investigate the role of TMIGD2 in AML, lentivirus-mediated shRNA was used to knockdown TMIGD2 on HEL cells. It was found that TMIGD2 knock-down enhanced both early apoptosis (Annexin V+DAPI−) and late apoptosis/necrosis (Annexin V+DAPI+) in HEL cells (FIG. 6A). To understand the molecular mechanisms by which TMIGD2 regulates AML function, RNA sequencing data generated from HEL-shCtrl and HEL-shTMIGD2 cells was analyzed. The analysis revealed that shTMIGD2 knock-down HEL cells, compared with shCtrl cells, were significantly enriched in genes involved in apoptosis and cell cycle arrest (FIG. 6B). These findings confirm that TMIGD2 is required for AML cell survival and proliferation.

### Treatment of Anti-TMIGD2 Monoclonal Antibodies Inhibit AML Progress In Vivo

To investigate the therapeutic efficacy of anti-TMIGD2 mAbs in AML in vivo, NSG mice were given AML cells from patients and then treated with anti-TMIGD2 mAbs 20F2 and 17C7. It was found that anti-TMIGD2 mAbs inhibited AML progression in vivo (FIG. 5D). These findings confirm that mAbs against TMIGD2 can be used to treat AML.

### REFERENCES

1. Janakiram, M., et al. The third group of the B7-CD28 immune checkpoint family: HHLA2, TMIGD2, B7x, and B7-H3. Immunol Rev 276, 26-39 (2017).
2. Zang, X. & Allison, J. P. The B7 family and cancer therapy: costimulation and coinhibition. Clin Cancer Res 13, 5271-5279 (2007).
3. John, P., et al. The B7x immune checkpoint pathway: From discovery to clinical trial. Trends Pharmacol Sci 40, 883-896 (2019).
4. Ohaegbulam, K. C., Assal, A., Lazar-Molnar, E., Yao, Y. & Zang, X. Human cancer immunotherapy with antibodies to the PD-1 and PD-L1 pathway. Trends Mol Med 21, 24-33 (2015).
5. Chinai, J. M., et al. New immunotherapies targeting the PD-1 pathway. Trends Pharmacol Sci 36, 587-595 (2015).
6. Vincenti, F., Dritselis, A. & Kirkpatrick, P. Belatacept. Nat Rev Drug Discov 10, 655-656 (2011).
7. Zhao, R., et al. HHLA2 is a member of the B7 family and inhibits human CD4 and CD8 T-cell function. Proc Natl Acad Sci USA 110, 9879-9884 (2013).
8. Janakiram, M., Chinai, J. M., Zhao, A., Sparano, J. A. & Zang, X. HHLA2 and TMIGD2: new immunotherapeutic targets of the B7 and CD28 families. Oncoimmunology 4, e1026534 (2015).
9. Janakiram, M., et al. Expression, clinical significance, and receptor identification of the newest B7 family member HHLA2 protein. Clin Cancer Res 21, 2359-2366 (2015).
10. Zhuang, X. & Long, E. O. CD28 Homolog Is a Strong Activator of Natural Killer Cells for Lysis of B7H7 (+) Tumor Cells. Cancer Immunol Res 7, 939-951 (2019).
11. Zhu, Y., et al. B7-H5 costimulates human T cells via CD28H. Nat Commun 4, 2043 (2013).
12. Wei, Y., et al. KIR3DL3-HHLA2 is a human immunosuppressive pathway and a cancer therapeutic target. Sci Immunol (2021). in Reversion
13. Zang, X. New immune checkpoint pathways: HHLA2 and its receptors including TMIGD2. Cold Spring Harbor Asia Conference on Precision Cancer Biology: From targeted immune therapies (2017).

The invention claimed is:

1. A method of treating or preventing cancer in a subject in need thereof, the method comprising administering to the subject an effective amount of an agent that inhibits TMIGD2 expression, activity, or both.

2. The method of claim 1, wherein the agent is selected from the group consisting of an antibody agent, a mRNA targeting agent, a small molecule agent, and a gene editing agent.

3. The method of claim 2, wherein the mRNA targeting agent is an antisense agent or an RNAi agent.

4. The method of claim 3, wherein the antisense agent comprises or consists of a nucleic acid sequence complementary to an mRNA encoded by SEQ ID NO: 4, SEQ ID NO:5, or SEQ ID NO:6, or the antisense agent comprises or consists of a nucleic acid sequence with about 80%, 90%, 95%, 96%, 97%, 98%, 99%, 99.5% or higher identity to an mRNA encoded by SEQ ID NO:4, SEQ ID NO:5, or SEQ ID NO:6.

5. The method of claim 3, wherein the RNAi agent is selected from the group consisting of a short interfering RNA (siRNA), a double-stranded RNA (dsRNA), a micro-RNA (miRNA), a piwi-RNA (piRNA), a small nucleolar RNA (snoRNA), a tRNA-derived small RNAs (tsRNAs), a small regulatory RNA (srRNA), and a short hairpin RNA (shRNA) molecule.

6. The method of claim 5, wherein the RNAi agent comprises or consists of a nucleic acid sequence complementary to an mRNA encoded by SEQ ID NO: 4, SEQ ID NO:5, or SEQ ID NO:6, or the RNAi agent comprises or consists of a nucleic acid sequence with about 80%, 90%, 95%, 96%, 97%, 98%, 99%, 99.5% or higher identity to an mRNA encoded by SEQ ID NO:4, SEQ ID NO:5, or SEQ ID NO:6.

7. The method of claim 2, wherein the gene editing agent is selected from the group consisting of a TALEN-based agent, ZFN-based agent, and a CRISPR-based agent.

8. The method of claim 7, wherein the gene editing agent knocks out or knocks down expression of TMIGD2.

9. The method of claim 2, wherein the antibody agent is an antibody or antigen-binding fragment thereof that specifically binds an epitope in the extracellular domain of TMIGD2.

10. The method of claim 9, wherein the extracellular domain of TMIGD2 comprises residues 1-150 of SEQ ID NO:1 or SEQ ID NO:2, or residues 1-30 of SEQ ID NO:3.

11. The method of claim 10, wherein the antibody or antigen-binding fragment thereof comprises:
(a) a heavy chain variable region comprising:
GYTFTSYDIN (SEQ ID NO: 24),
WIYPGDGSTNYNEKFKG (SEQ ID NO: 25), and
ARRGLRYYFDY (SEQ ID NO: 26); and/or
a light chain variable region comprising:
RASQDIRNYLN (SEQ ID NO: 32),
YTSRLHS (SEQ ID NO: 33), and
QQVNTLPWT (SEQ ID NO: 34); or
(b) a heavy chain variable region comprising:
GYSITSDYAWN (SEQ ID NO: 56).
YITYSGSTSYNPSLKS (SEQ ID NO: 57), and
ARSGYRYDDAMDY (SEQ ID NO: 58); and/or
a light chain variable region comprising:
KSSQSLLSSNNQKNYLA (SEQ ID NO: 64),
FASTRES (SEQ ID NO: 65), and
QQHYRTPLT (SEQ ID NO: 66).

12. The method of claim 11, wherein the antibody or antigen-binding fragment thereof comprises:
(a) a heavy chain variable region comprising SEQ ID NO: 23; and/or a light chain variable region comprising SEQ ID NO: 31; or
(b) a heavy chain variable region comprising SEQ ID NO: 55; and/or a light chain variable region comprising SEQ ID NO: 63.

13. The method of claim 12, wherein the cancer is a human hematologic malignancy.

14. The method of claim 13, wherein the human hematologic malignancy is selected from myeloid neoplasm, acute myeloid leukemia (AML), AML with recurrent genetic abnormalities, AML with myelodysplasia-related changes, therapy-related AML, acute leukemias of ambiguous lineage, myeloproliferative neoplasm, essential thrombocythemia, polycythemia vera, myelofibrosis (MF), primary myelofibrosis, systemic mastocytosis, myelodysplastic syndromes (MDS), myeloproliferative/myelodysplastic syndromes, chronic myeloid leukemia, chronic neutrophilic leukemia, chronic eosinophilic leukemia, myelodysplastic syndromes (MDS), refractory anemia with ringed sideroblasts, refractory cytopenia with multilineage dysplasia, refractory anemia with excess blasts (type 1), refractory anemia with excess blasts (type 2), MDS with isolated del (5q), unclassifiable MDS, myeloproliferative/myelodysplastic syndromes, chronic myelomonocytic leukemia, atypical chronic myeloid leukemia, juvenile myelomonocytic leukemia, unclassifiable myeloproliferative/myelodysplatic syndromes, lymphoid neoplasms, precursor lymphoid neoplasms, B lymphoblastic leukemia, B lymphoblastic lymphoma, T lymphoblastic leukemia, T lymphoblastic lymphoma, mature B-cell neoplasms, diffuse large B-cell lymphoma, primary central nervous system lymphoma, primary mediastinal B-cell lymphoma, Burkitt lymphoma/leukemia, follicular lymphoma, chronic lymphocytic leukemia, small lymphocytic lymphoma, B-cell prolymphocytic leukemia, lymphoplasmacytic lymphoma, Waldenström macroglobulinemia, mantle cell lymphoma, marginal zone lymphomas, post-transplant lymphoproliferative disorders, HIV-associated lymphomas, primary effusion lymphoma, intravascular large B-cell lymphoma, primary cutaneous B-cell lymphoma, hairy cell leukemia, multiple myeloma, monoclonal gammopathy of unknown significance (MGUS), smoldering multiple myeloma, or solitary plasmacytomas (solitary bone and extramedullary).

15. An anti-TMIGD2 antibody or antigen-binding fragment thereof comprising:
(a) a heavy chain variable region comprising:
GYTFTSYDIN (SEQ ID NO: 24),
WIYPGDGSTNYNEKFKG (SEQ ID NO: 25), and
ARRGLRYYFDY (SEQ ID NO: 26); and/or
a light chain variable region comprising:
RASQDIRNYLN (SEQ ID NO: 32),
YTSRLHS (SEQ ID NO: 33), and
QQVNTLPWT (SEQ ID NO: 34); or
(b) a heavy chain variable region comprising:
GYSITSDYAWN (SEQ ID NO: 56).
YITYSGSTSYNPSLKS (SEQ ID NO: 57), and
ARSGYRYDDAMDY (SEQ ID NO: 58); and/or
a light chain variable region comprising:
KSSQSLLSSNNQKNYLA (SEQ ID NO: 64),
FASTRES (SEQ ID NO: 65), and
QQHYRTPLT (SEQ ID NO: 66).

16. The anti-TMIGD2 antibody or antigen-binding fragment thereof of claim 15, wherein the antibody comprises:
(a) a heavy chain variable region comprising SEQ ID NO: 23; and/or a light chain variable region comprising SEQ ID NO: 31; or
(b) a heavy chain variable region comprising SEQ ID NO: 55; and/or a light chain variable region comprising SEQ ID NO: 63.
